SEQUENCE LISTING (2 sequences)

SEQ ID NO: 1 - protein, 133 aa, Homo sapiens
  PEPTIDE (1)..(133): human interleukin-2 (IL-2) polypeptide
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 2 - protein, 133 aa, Homo sapiens
  PEPTIDE (1)..(133): human interleukin-2 (IL-2) polypeptide
MPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

(19) United States
(12) Patent Application Publication
(10) Pub. No.: US 2022/0289806 A1
(43) Pub. Date: Sep. 15, 2022

(54) Modified Interleukin 2 (IL-2) Polypeptides, Conjugates and Uses Thereof

(71) Applicant: Cytimm Therapeutics, Inc., San Diego, CA (US)

(72) Inventors: Xiao XU, San Diego, CA (US); Haining HUANG, San Diego, CA (US); YU FENG, San Diego, CA (US); GUILIANA MOGNOL, San Diego, CA (US); CAN JIN, San Diego, CA (US); DIANA GUIMET, San Diego, CA (US)

(21) Appl. No.: 17/634,022

(22) PCT Filed: Aug. 11, 2020
(86) PCT No.: PCT/US2020/045810

§ 371 Date: Feb. 9, 2022

Related U.S. Application Data
(60) Provisional application No. 62/887,359, filed on Aug. 15, 2019.
(60) Provisional application No. 63/025,095, filed on May 14, 2020.

(51) Int. Cl. C07K 14/55; C12N 5/0783; A61K 47/02; A61K 47/54; A61K 47/42; A61P 35/00

(52) U.S. Cl. CPC C07K 14/55; C12N 5/0646; A61K 47/02; A61K 47/543; A61K 47/42; A61P 35/00

(57) ABSTRACT

This disclosure relates to modified interleukin 2 (IL-2) polypeptides, polynucleotides, e.g., DNA, RNA or viral vector, that encode the modified IL-2 polypeptides and are configured to express said modified IL-2 polypeptides in vitro and/or in vivo, conjugates comprising the modified IL-2 polypeptides, and uses thereof.

### CROSS REFERENCE TO RELATED APPLICATIONS

This application claims priority to U.S. provisional patent application No. 62/887,359, filed on Aug. 15, 2019, entitled “Modified Interleukin 2 (IL-2) Polypeptides, Conjugates And Uses Thereof,” and U.S. provisional patent application No. 63/025,095, filed on May 14, 2020, entitled “Modified Interleukin 2 (IL-2) Polypeptides, Conjugates And Uses Thereof” The contents and disclosures of the above applications are incorporated herein by reference in their entireties for all purposes.

### SEQUENCE LISTING ON ASCII TEXT

This patent or application file contains a Sequence Listing submitted in computer readable ASCII text format (file name: 7006-2000140_SeqList_ST25.txt, date recorded: Aug. 10, 2020, size: 3,014 bytes). The content of the Sequence Listing file is incorporated herein by reference in its entirety.

### TECHNICAL FIELD

This disclosure relates to modified interleukin 2 (IL-2) polypeptides, with or without conjugates comprising the modified IL-2 polypeptides, polynucleotides, e.g., DNA, RNA or viral vector, that encode the modified IL-2 polypeptide and are configured to express said modified IL-2 polypeptide in vitro and/or in vivo, and uses thereof.

### BACKGROUND ART

Clinical use of interleukin-2 (IL-2) for cancer treatment has been mainly limited by toxicity and short half-life in vivo [1,2]. It was observed that the toxicity was markedly reduced in animals deficient in CD25 (IL-2 receptor α unit, IL-2Rα) [3]. PEGylation, the covalent attachment of Polyethylene glycol (PEG) to therapeutics, has been shown to overcome obstacles such as rapid body clearance, aggregation and enzymatic degradation [4].

WO 2019/028419 A1 and WO 2019/028425 A1 disclose interleukin (IL) conjugates (e.g., IL-2 conjugates) and use in the treatment of one or more indications. Also described in WO 2019/028419 A 1 and WO 2019/028425 are pharmaceutical compositions and kits comprising one or more of the interleukin conjugates (e.g., IL-2 conjugates).

There exists in the art a need for improved modified interleukin 2 (IL-2) polypeptides with or without conjugates. The present invention addresses this and other related needs in the art.

### SUMMARY

The present invention is directed to modified interleukin 2 (IL-2) polypeptides, polynucleotides, e.g., DNA, RNA or viral vector, that encode the modified IL-2 polypeptide and are configured to express said modified IL-2 polypeptide in vitro and/or in vivo, conjugates comprising the modified IL-2 polypeptides, and uses thereof.

In one aspect, the present invention is directed to a modified interleukin 2 (IL-2) polypeptide, which comprises an amino acid sequence set forth in SEQ ID NO:1 or SEQ ID NO:2 and a substitution with a natural amino acid or an unnatural amino acid at a position selected from the group consisting of Q13, L19, N29, N30, Y31, K32, N33, P34, K35, T37, R38, T41, F42, K43, Y45, K48, K49, E62, K64, P65, N71, Q74, K76, R81, L85, S87, V91, I92, V93 and a combination thereof, wherein said modified IL-2 polypeptide: a) is configured to be unconjugated or conjugated to a water-soluble polymer, a lipid, or a polypeptide, e.g., a protein or a peptide; b) has reduced binding to an interleukin 2 receptor α (IL-2Rα) compared to a comparable IL-2 polypeptide comprising an amino acid sequence set forth in SEQ ID NO:1 or SEQ ID NO:2 without the substitution; c) has reduced receptor signaling potency to IL-2Rαβγ compared to a comparable IL-2 polypeptide comprising an amino acid sequence set forth in SEQ ID NO:1 or SEQ ID NO:2 without the substitution, d) has increased ratio of signaling potency to IL-2Rβγ over signaling potency to IL-2Rαβγ (i.e., increased ratio of signaling potency to IL-2Rβγ/signaling potency to IL-2Rαβγ) compared to a comparable IL-2 polypeptide comprising an amino acid sequence set forth in SEQ ID NO:1 or SEQ ID NO:2 without the substitution, and/or e) has enhanced receptor signaling potency to IL-2Rβγ compared to a comparable IL-2 polypeptide comprising an amino acid sequence set forth in SEQ ID NO:1 or SEQ ID NO:2 without the substitution, and provided that when said modified IL-2 polypeptide comprises a substitution with an unnatural amino acid, said modified IL-2 polypeptide comprises a substitution at a position selected from the group consisting of N29, N30, Y31, K32, N33, P34, K35, R38, T41, F42, K43, Y45, K48, K49, E62, K64, P65, N71, Q74, K76 and a combination thereof, and a substitution with a natural amino acid or an unnatural amino acid at a position within IL-2Rα interaction region, IL-2Rβ interaction region and/or IL-2Rγ interaction region, and provided that said modified IL-2 polypeptide has at least about 80% sequence identity in the region of amino acid residues 10-25, 80-100 and/or 100-134 to the corresponding region of a comparable IL-2 polypeptide comprising an amino acid sequence set forth in SEQ ID NO:1 or SEQ ID NO:2 without the substitution, and said modified IL-2 polypeptide has at least about 50% sequence identity to a comparable IL-2 polypeptide comprising an amino acid sequence set forth in SEQ ID NO:1 or SEQ ID NO:2 without the substitution.

In another aspect, the present invention is directed to a polynucleotide, e.g., DNA, RNA or viral vector, that encodes the modified IL-2 polypeptide and is configured to express said modified IL-2 polypeptide in vitro and/or in vivo. In some embodiments, the modified IL-2 polypeptide, as described above, with or without conjugate, can be applied in the format of a protein, a fusion protein, a protein conjugate, or as part of nanoparticles. In some embodiments, the above polynucleotide, e.g., DNA, RNA or viral vector, that encodes the modified IL-2 polypeptide and is configured to express said modified IL-2 polypeptide in vitro and/or in vivo, can be applied to cell(s), tissue(s), organ(s), or subject(s), e.g., human subject(s).

In still another aspect, the present invention is directed to a modified IL-2 polypeptide conjugate, which comprises a modified IL-2 polypeptide, as described above, that is conjugated to a water-soluble polymer, a lipid, a polypeptide, e.g., a protein, or a peptide.

In yet another aspect, the present invention is directed to a pharmaceutical composition comprising an effective amount of a modified IL-2 polypeptide, a polynucleotide, e.g., DNA, RNA or viral vector, or a modified IL-2 polypeptide conjugate, as described above, and a pharmaceutically acceptable carrier or excipient.

In yet another aspect, the present invention is directed to a method for treating or preventing a disease or a disorder, e.g., a proliferation disease or disorder, an autoimmune or inflammatory disease or disorder, or an infectious disease or disorder, in a subject in need comprising administering to said subject an effective amount of a modified IL-2, a polynucleotide, e.g., DNA, RNA or viral vector, a modified IL-2 polypeptide conjugate or a pharmaceutical composition, as described above.

In yet another aspect, the present invention is directed to an use of an effective amount of a modified IL-2 polypeptide, a polynucleotide, e.g., DNA, RNA or viral vector, or a modified IL-2 polypeptide conjugate, as described above, for the manufacture of a medicament for treating or preventing a disease or a disorder, e.g., a proliferation disease or disorder, an autoimmune or inflammatory disease or disorder, or an infectious disease or disorder, in a subject.

In yet another aspect, the present invention is directed to a method of expanding a CD4⁺ helper cell, CD8⁺ effector naive and memory cell, Natural Killer (NK) cell, or Natural killer T (NKT) cell population, which comprises contacting a cell population with an effective amount of a modified IL-2 polypeptide, a polynucleotide, e.g., DNA, RNA or viral vector, or a modified IL-2 polypeptide conjugate, as described above, or a pharmaceutical composition comprising the modified IL-2 polypeptide, a polynucleotide, e.g., DNA, RNA or viral vector, or modified IL-2 polypeptide conjugate for a time sufficient to induce formation of a complex with an IL-2Rβγ, thereby stimulating the expansion of the T cell, NK cell, and/or NKT cell population.

In yet another aspect, the present invention is directed to a method of expanding a CD4⁺ helper cell, CD8⁺ effector naive and memory cell, Treg cells, Natural Killer (NK) cell, or Natural killer T (NKT) cell population, which comprises contacting a cell population with an effective amount of a modified IL-2 polypeptide, a polynucleotide, e.g., DNA, RNA or viral vector, or a modified IL-2 polypeptide conjugate, as described above, or a pharmaceutical composition comprising the modified IL-2 polypeptide, a polynucleotide, e.g., DNA, RNA or viral vector, or modified IL-2 polypeptide conjugate for a time sufficient to induce formation of a complex with an IL-2Rβγ, thereby stimulating the expansion of the T cell, Treg cell, NK cell, and/or NKT cell population with reduced cell death by 10% to 100%.

In yet another aspect, the present invention is directed to an use of an effective amount of a modified IL-2 polypeptide, a polynucleotide, e.g., DNA, RNA or viral vector, or a modified IL-2 polypeptide conjugate, as described above, for the manufacture of a medicament for expanding a CD4⁺ helper cell, CD8⁺ effector naive and memory cell, Treg cell, Natural Killer (NK) cell, or Natural killer T (NKT) cell in a cell population.

Other aspects and advantages of the present invention will be apparent from the embodiments and examples provided herein.

For the sake of brevity, the disclosures of the publications cited in this specification, including patents, are herein incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A illustrates sequence of an exemplary recombinant human IL-2 with mutation from Cysteine to Serine at position 125 (rhIL-2) [5]. The amino acid sites selected to be PEGylated through individual cysteine substitution and/or the sites selected to disrupt IL-2Rα interaction and/or enhance IL-2Rβγ interaction by mutation are labeled by superscripted numbers. FIG. 1B illustrates 3-D structure of IL-2 and receptor IL-2Rαβγ complex which derived from PDB structure 2b5i. See e.g., The Protein Data Bank H. M. Berman, J. Westbrook, Z. Feng, G. Gilliland, T. N. Bhat, H. Weissig, I. N. Shindyalov, P. E. Bourne (2000) Nucleic Acids Research, 28: 235-242. doi:10.1093/nar/28.1.235. The sites described in FIG. 1A are shown as red spheres.

FIG. 2 illustrates that expression of exemplary functional IL-2 variants with individual cysteine substitution was determined by HEK-blue assay at 1:10000 diluted cell culture supernatant.

FIG. 3 illustrates exemplary or typical profile of chromatography and SDS-PAGE analysis for exemplary IL-2 muteins and PEG-conjugates. FIG. 3A shows chromatography of N29C by Superdex 75 Increase column. FIG. 3B shows chromatography of N29C-PEG30 conjugate by SP Sepharose FF column. FIG. 3C shows chromatography of N29C-PEG30 conjugate by Superdex 75 Increase column. FIG. 3D shows SDS-PAGE analysis of N29C-PEG30 fractions eluted from SP Sepharose FF column and followed Superdex 75 Increase column.

FIG. 4 illustrates exemplary or representative sensorgrams of exemplary IL-2 muteins and PEG-conjugates binding with IL-2Rα obtained by Octet Qke (ForteBio, San Jose, Calif.).

FIG. 5 illustrates that Y31C mutation and pegylation did not affect cytokine binding with IL2Rα obtained by demonstrated by Octet Qke (ForteBio, San Jose, Calif.).

FIG. 6 illustrates that Y31C mutein has enhanced binding on IL2Rαβγ expressing cells like CTLL2 cells and CD25+ human T cells.

FIG. 7 illustrates activation of pSTAT5 by exemplary IL-2 muteins and PEG-conjugates in human T cell subpopulations.

FIG. 8 illustrates plot of the concentration-time curves following a single injection of rhIL-2, P65C-PEG20 conjugate and Y31C-PEG20+F42K conjugate in mice.

FIG. 9 illustrate that rhIL-2, P65C-PEG20 conjugate and Y31C-PEG20+F42K conjugate stimulate ex vivo expansion of T cells and NK cells.

FIG. 10 illustrates that rhIL-2, P65C-PEG20 conjugate and Y31C-PEG20+F42K conjugate amplified LAK cells (NK cells) have enhanced cytotoxicity.

### DETAILED DESCRIPTION

### A. General Techniques

The practice of the present invention will employ, unless otherwise indicated, conventional techniques of molecular biology (including recombinant techniques), microbiology, cell biology, biochemistry, immunology, and pharmacology, which are within the skill of the art. Such techniques are explained fully in the literature, such as, Molecular Cloning: A Laboratory Manual, 2^{nd} ed. (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction (Mullis et al., eds., 1994); and Remington, The Science and Practice of Pharmacy, 20^{th} ed., (Lippincott, Williams & Wilkins 2003).

### B. Definitions

Unless defined otherwise, all technical and scientific terms used herein have the same meaning as is commonly understood by one of ordinary skill in the art to which this invention belongs. All patents, patent applications (published or unpublished), and other publications referred to herein are incorporated by reference in their entireties. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

The terms “polypeptide,” “oligopeptide,” “peptide,” and “protein” are used interchangeably herein to refer to polymers of amino acids of any length, e.g., at least 5, 6, 7, 8, 9, 10, 20, 30, 40, 50, 100, 200, 300, 400, 500, 1,000 or more amino acids. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, the terms “variant” is used in reference to polypeptides that have some degree of amino acid sequence identity to a parent polypeptide sequence. A variant is similar to a parent sequence, but has at least one substitution, deletion or insertion in their amino acid sequence that makes them different in sequence from a parent polypeptide. Additionally, a variant may retain the functional characteristics of the parent polypeptide, e.g., maintaining a biological activity that is at least 50%, 60%, 70%, 80%, 90%, 95%, 98%, or 99% of that of the parent polypeptide.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule, and can be an immunoglobulin of any class, e.g., IgG, IgM, IgA, IgD and IgE. IgY, which is the major antibody type in avian species such as chicken, is also included within the definition. As used herein, the term encompasses not only intact polyclonal or monoclonal antibodies, but also fragments thereof (such as Fab, Fab′, F(ab′)2, Fv), single chain (ScFv), mutants thereof, naturally occurring variants, fusion proteins comprising an antibody portion with an antigen recognition site of the required specificity, humanized antibodies, chimeric antibodies, and any other modified configuration of the immunoglobulin molecule that comprises an antigen recognition site of the required specificity.

As used herein, the term “antigen” refers to a target molecule that is specifically bound by an antibody through its antigen recognition site. The antigen may be monovalent or polyvalent, i.e., it may have one or more epitopes recognized by one or more antibodies. Examples of kinds of antigens that can be recognized by antibodies include polypeptides, oligosaccharides, glycoproteins, polynucleotides, lipids, etc.

As used herein, the term “epitope” refers to a portion of an antigen, e.g., a peptide sequence of at least about 3 to 5, preferably about 5 to 10 or 15, and not more than about 1,000 amino acids (or any integer there between), which define a sequence that by itself or as part of a larger sequence, binds to an antibody generated in response to such sequence. There is no critical upper limit to the length of the fragment, which may, for example, comprise nearly the full-length of the antigen sequence, or even a fusion protein comprising two or more epitopes from the target antigen. An epitope for use in the subject invention is not limited to a peptide having the exact sequence of the portion of the parent protein from which it is derived, but also encompasses sequences identical to the native sequence, as well as modifications to the native sequence, such as deletions, additions and substitutions (conservative in nature).

As used herein, the term “specifically binds” refers to the binding specificity of a specific binding pair. Recognition by an antibody of a particular target in the presence of other potential targets is one characteristic of such binding. Specific binding involves two different molecules wherein one of the molecules specifically binds with the second molecule through chemical or physical means. The two molecules are related in the sense that their binding with each other is such that they are capable of distinguishing their binding partner from other assay constituents having similar characteristics. The members of the binding component pair are referred to as ligand and receptor (anti-ligand), specific binding pair (SBP) member and SBP partner, and the like. A molecule may also be an SBP member for an aggregation of molecules; for example an antibody raised against an immune complex of a second antibody and its corresponding antigen may be considered to be an SBP member for the immune complex.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping groups moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR 2 (“amidate”), P(O)R, P(O)OR′, CO or CH 2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

“Oligonucleotide,” as used herein, generally refers to short, generally single stranded, generally synthetic polynucleotides that are generally, but not necessarily, less than about 200 nucleotides in length. The terms “oligonucleotide” and “polynucleotide” are not mutually exclusive. The description above for polynucleotides is equally and fully applicable to oligonucleotides.

As used herein, the term “homologue” is used to refer to a nucleic acid which differs from a naturally occurring nucleic acid (e.g., the “prototype” or “wild-type” nucleic acid) by minor modifications to the naturally occurring nucleic acid, but which maintains the basic nucleotide structure of the naturally occurring form. Such changes include, but are not limited to: changes in one or a few nucleotides, including deletions (e.g., a truncated version of the nucleic acid) insertions and/or substitutions. A homologue can have enhanced, decreased, or substantially similar properties as compared to the naturally occurring nucleic acid. A homologue can be complementary or matched to the naturally occurring nucleic acid. Homologues can be produced using techniques known in the art for the production of nucleic acids including, but not limited to, recombinant DNA techniques, chemical synthesis, etc.

As used herein, “substantially complementary or substantially matched” means that two nucleic acid sequences have at least 90% sequence identity. Preferably, the two nucleic acid sequences have at least 95%, 96%, 97%, 98%, 99% or 100% of sequence identity. Alternatively, “substantially complementary or substantially matched” means that two nucleic acid sequences can hybridize under high stringency condition(s).

In general, the stability of a hybrid is a function of the ion concentration and temperature. Typically, a hybridization reaction is performed under conditions of lower stringency, followed by washes of varying, but higher, stringency. Moderately stringent hybridization refers to conditions that permit a nucleic acid molecule such as a probe to bind a complementary nucleic acid molecule. The hybridized nucleic acid molecules generally have at least 60% identity, including for example at least any of 70%, 75%, 80%, 85%, 90%, or 95% identity. Moderately stringent conditions are conditions equivalent to hybridization in 50% formamide, 5×Denhardt's solution, 5×SSPE, 0.2% SDS at 42° C., followed by washing in 0.2×SSPE, 0.2% SDS, at 42° C. High stringency conditions can be provided, for example, by hybridization in 50% formamide, 5×Denhardt's solution, 5×SSPE, 0.2% SDS at 42° C., followed by washing in 0.1×SSPE, and 0.1% SDS at 65° C. Low stringency hybridization refers to conditions equivalent to hybridization in 10% formamide, 5×Denhardt's solution, 6×SSPE, 0.2% SDS at 22° C., followed by washing in 1×SSPE, 0.2% SDS, at 37° C. Denhardt's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M (EDTA). Other suitable moderate stringency and high stringency hybridization buffers and conditions are well known to those of skill in the art.

As used herein, “vector (or plasmid)” refers to discrete elements that are used to introduce heterologous DNA into cells for either expression or replication thereof. Selection and use of such vehicles are well known within the skill of the artisan. An expression vector includes vectors capable of expressing DNA's that are operatively linked with regulatory sequences, such as promoter regions, that are capable of effecting expression of such DNA fragments. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

As used herein, “a promoter region or promoter element” refers to a segment of DNA or RNA that controls transcription of the DNA or RNA to which it is operatively linked. The promoter region includes specific sequences that are sufficient for RNA polymerase recognition, binding and transcription initiation. This portion of the promoter region is referred to as the promoter. In addition, the promoter region includes sequences that modulate this recognition, binding and transcription initiation activity of RNA polymerase. These sequences may be cis acting or may be responsive to trans acting factors. Promoters, depending upon the nature of the regulation, may be constitutive or regulated. Exemplary promoters contemplated for use in prokaryotes include the bacteriophage T7 and T3 promoters, and the like.

As used herein, “operatively linked or operationally associated” refers to the functional relationship of DNA with regulatory and effector sequences of nucleotides, such as promoters, enhancers, transcriptional and translational stop sites, and other signal sequences. For example, operative linkage of DNA to a promoter refers to the physical and functional relationship between the DNA and the promoter such that the transcription of such DNA is initiated from the promoter by an RNA polymerase that specifically recognizes, binds to and transcribes the DNA. In order to optimize expression and/or in vitro transcription, it may be necessary to remove, add or alter 5′ untranslated portions of the clones to eliminate extra, potential inappropriate alternative translation initiation (i.e., start) codons or other sequences that may interfere with or reduce expression, either at the level of transcription or translation. Alternatively, consensus sites can be inserted immediately 5′ of the start codon and may enhance expression. See, e.g., Kozak (1991) J. Biol. Chem. 266:19867-19870. The desirability of (or need for) such modification may be empirically determined.

“Treating” or “treatment” or “alleviation” refers to therapeutic treatment wherein the object is to slow down (lessen) if not cure the targeted pathologic condition or disorder or prevent recurrence of the condition. A subject is successfully “treated” if, after receiving a therapeutic amount of a therapeutic agent or treatment, the subject shows observable and/or measurable reduction in or absence of one or more signs and symptoms of the particular disease. Reduction of the signs or symptoms of a disease may also be felt by the patient. A patient is also considered treated if the patient experiences stable disease. In some embodiments, treatment with a therapeutic agent is effective to result in the patients being disease-free 3 months after treatment, preferably 6 months, more preferably one year, even more preferably 2 or more years post treatment. These parameters for assessing successful treatment and improvement in the disease are readily measurable by routine procedures familiar to a physician of appropriate skill in the art. In some embodiments, “treatment” means any manner in which the symptoms of a condition, disorder or disease are ameliorated or otherwise beneficially altered. Treatment also encompasses any pharmaceutical use of the compositions herein. In some embodiments, “amelioration” of the symptoms of a particular disorder by administration of a particular pharmaceutical composition refers to any lessening, whether permanent or temporary, lasting or transient that can be attributed to or associated with administration of the composition.

The term “prediction” or “prognosis” is often used herein to refer to the likelihood that a patient will respond either favorably or unfavorably to a drug or set of drugs, or the likely outcome of a disease. In one embodiment, the prediction relates to the extent of those responses or outcomes. In one embodiment, the prediction relates to whether and/or the probability that a patient will survive or improve following treatment, for example treatment with a particular therapeutic agent, and for a certain period of time without disease recurrence. The predictive methods of the invention can be used clinically to make treatment decisions by choosing the most appropriate treatment modalities for any particular patient. The predictive methods of the present invention are valuable tools in predicting if a patient is likely to respond favorably to a treatment regimen, such as a given therapeutic regimen, including for example, administration of a given therapeutic agent or combination, surgical intervention, steroid treatment, etc.

As used herein the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

A “pharmaceutically acceptable salt” is intended to mean a salt of a free acid or base of a compound represented herein that is non-toxic, biologically tolerable, or otherwise biologically suitable for administration to the subject. See, generally, Berge, et al., J. Pharm. Sci., 1977, 66, 1-19. Preferred pharmaceutically acceptable salts are those that are pharmacologically effective and suitable for contact with the tissues of subjects without undue toxicity, irritation, or allergic response. A modified interleukin 2 (IL-2) polypeptide or its conjugate described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt.

Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates.

As used herein, the term “therapeutically effective amount” or “effective amount” refers to an amount of a therapeutic agent that when administered alone or in combination with an additional therapeutic agent to a cell, tissue, or subject is effective to prevent or ameliorate a disease or disorder, a proliferation disease or disorder, in a subject. A therapeutically effective dose further refers to that amount of the therapeutic agent sufficient to result in amelioration of symptoms, e.g., treatment, healing, prevention or amelioration of the relevant medical condition, or an increase in rate of treatment, healing, prevention or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. In some embodiment, “an effective amount of a compound for treating a particular disease” is an amount that is sufficient to ameliorate, or in some manner reduce the symptoms associated with the disease. Such amount may be administered as a single dosage or may be administered according to a regimen, whereby it is effective. The amount may cure the disease but, typically, is administered in order to ameliorate the symptoms of the disease. Repeated administration may be required to achieve the desired amelioration of symptoms.

The term “combination” refers to either a fixed combination in one dosage unit form, or a kit of parts for the combined administration where a modified interleukin 2 (IL-2) polypeptide or its conjugate and a combination partner (e.g., another drug as explained below, also referred to as “therapeutic agent” or “co-agent”) may be administered independently at the same time or separately within time intervals, especially where these time intervals allow that the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a modified interleukin 2 (IL-2) polypeptide or its conjugate and a combination partner, are both administered to a patient simultaneously in the form of a single entity or dosage. The term “non-fixed combination” means that the active ingredients, e.g., a modified interleukin 2 (IL-2) polypeptide or its conjugate and a combination partner, are both administered to a patient as separate entities either simultaneously, concurrently or sequentially with no specific time limits, wherein such administration provides therapeutically effective levels of the two substances in the body of the patient. The latter also applies to cocktail therapy, e.g., the administration of three or more active ingredients.

As used herein, “biological sample” refers to any sample obtained from a living or viral source or other source of macromolecules and biomolecules, and includes any cell type or tissue of a subject from which nucleic acid or protein or other macromolecule can be obtained. The biological sample can be a sample obtained directly from a biological source or a sample that is processed. For example, isolated nucleic acids that are amplified constitute a biological sample. Biological samples include, but are not limited to, body fluids, such as blood, plasma, serum, cerebrospinal fluid, synovial fluid, urine and sweat, tissue and organ samples from animals and plants and processed samples derived therefrom.

The terms “level” or “levels” are used to refer to the presence and/or amount of a target, e.g., a substance or an organism that is part of the etiology of a disease or disorder, and can be determined qualitatively or quantitatively. A “qualitative” change in the target level refers to the appearance or disappearance of a target that is not detectable or is present in samples obtained from normal controls. A “quantitative” change in the levels of one or more targets refers to a measurable increase or decrease in the target levels when compared to a healthy control.

A “healthy control” or “normal control” is a biological sample taken from an individual who does not suffer from a disease or disorder, e.g., a proliferation disease or disorder. A “negative control” is a sample that lacks any of the specific analyte the assay is designed to detect and thus provides a reference baseline for the assay.

As used herein, “mammal” refers to any of the mammalian class of species. Frequently, the term “mammal,” as used herein, refers to humans, human subjects or human patients. “Mammal” also refers to any of the non-human mammalian class of species, e.g., experimental, companion or economic non-human mammals. Exemplary non-human mammals include mice, rats, rabbits, cats, dogs, pigs, cattle, sheep, goats, horses, monkeys, Gorillas and chimpanzees.

As used herein, “production by recombinant means” refers to production methods that use recombinant nucleic acid methods that rely on well-known methods of molecular biology for expressing polypeptides or proteins encoded by cloned nucleic acids.

As used herein, the term “subject” is not limited to a specific species or sample type. For example, the term “subject” may refer to a patient, and frequently a human patient. However, this term is not limited to humans and thus encompasses a variety of non-human animal or mammalian species.

As used herein, a “prodrug” is a substance that, upon in vivo administration, is metabolized or otherwise converted to the biologically, pharmaceutically or therapeutically active form of the substance. To produce a prodrug, the pharmaceutically active substance is modified such that the active substance will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

It is understood that aspects and embodiments of the invention described herein include “consisting” and/or “consisting essentially of” aspects and embodiments.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Other objects, advantages and features of the present invention will become apparent from the following specification taken in conjunction with the accompanying drawings.

### C. Modified Interleukin 2 (IL-2) Polypeptides and Polynucleotides Encoding and Expressing the Same

In one aspect, the present invention is directed to a modified interleukin 2 (IL-2) polypeptide, which comprises an amino acid sequence set forth in SEQ ID NO:1 or SEQ ID NO:2 and a substitution with a natural amino acid or an unnatural amino acid at a position of Q13, L19, N29, N30, Y31, K32, N33, P34, K35, T37, R38, T41, F42, K43, Y45, K48, K49, E62, K64, P65, N71, Q74, K76, R81, L85, S87, V91, I92, V93, or a combination thereof, wherein said modified IL-2 polypeptide: a) is configured to be conjugated to a water-soluble polymer, a lipid, or a polypeptide, e.g., a protein or a peptide; b) has reduced binding to an interleukin 2 receptor α (IL-2Rα) compared to a comparable IL-2 polypeptide comprising an amino acid sequence set forth in SEQ ID NO:1 or SEQ ID NO:2 without the substitution; and/or c) has reduced receptor signaling potency to IL-2Rαβγ compared to a comparable IL-2 polypeptide comprising an amino acid sequence set forth in SEQ ID NO:1 or SEQ ID NO:2 without the substitution, and provided that when said modified IL-2 polypeptide comprises a substitution with an unnatural amino acid, said modified IL-2 polypeptide comprises a substitution at a of N29, N30, Y31, K32, N33, P34, K35, R38, T41, F42, K43, Y45, K48, K49, E62, K64, P65, N71, Q74, K76 or a combination thereof, and a substitution with a natural amino acid or an unnatural amino acid at a position within IL-2Rα interaction region, IL-2Rβ interaction region and/or IL-2Rγ interaction region, and provided that said modified IL-2 polypeptide has at least about 80% sequence identity in the region of amino acid residues 10-25, 80-100 and/or 100-134 to the corresponding region of a comparable IL-2 polypeptide comprising an amino acid sequence set forth in SEQ ID NO:1 or SEQ ID NO:2 without the substitution, and said modified IL-2 polypeptide has at least about 50% sequence identity to a comparable IL-2 polypeptide comprising an amino acid sequence set forth in SEQ ID NO:1 or SEQ ID NO:2 without the substitution.

The amino acid sequences of SEQ ID NO:1 or SEQ ID NO:2 are set forth below:

SEQ ID NO: 1
(¹APTSSSTKKTQL¹³QLEHLL¹⁹LDLQMILNGI²⁹N³⁰N³¹Y³²K³³N
³⁴P³⁵KLT³⁸RML⁴¹T⁴²F⁴³KF⁴⁵YMP⁴⁸K⁴⁹KATELKHLQCLEE⁶²EL
⁶⁴K⁶⁵PLEEVL⁷¹NLA⁷⁴QS⁷⁶KNFHL⁸¹RPRD⁸⁵LI⁸⁷SNIN⁹¹V⁹²I
⁹³VLELKGSETTFMCEYADETATIVEFLNRWITFSQSIISTL¹³³T)
SEQ ID NO: 2
(¹MPTSSSTKKTQL¹³QLEHLL¹⁹LDLQMILNGI²⁹N³⁰N³¹Y³²K³³N
³⁴P³⁵KLT³⁸RML⁴¹T⁴²F⁴³KF⁴⁵YMP⁴⁸K⁴⁹KATELKHLQCLEE⁶²EL
⁶⁴K⁶⁵PLEEVL⁷¹NLA⁷⁴QS⁷⁶KNFHL⁸¹RPRD⁸⁵LI⁸⁷SNIN⁹¹V⁹²I
⁹³VLELKGSETTFMCEYADETATIVEFLNRWITFCQSIISTL¹³³T)

In one embodiment, the modified IL-2 polypeptide has at least about 80% sequence identity, e.g., at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity in the region of amino acid residues 10-25 to the corresponding region of a comparable IL-2 polypeptide comprising an amino acid sequence set forth in SEQ ID NO:1 or SEQ ID NO:2 without the substitution.

In another embodiment, the modified IL-2 polypeptide has at least about 80% sequence identity, e.g., at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity in the region of amino acid residues 80-100 to the corresponding region of a comparable IL-2 polypeptide comprising an amino acid sequence set forth in SEQ ID NO:1 or SEQ ID NO:2 without the substitution.

In still another embodiment, the modified IL-2 polypeptide has at least about 80% sequence identity, e.g., at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity in the region of amino acid residues 100-134 to the corresponding region of a comparable IL-2 polypeptide comprising an amino acid sequence set forth in SEQ ID NO:1 or SEQ ID NO:2 without the substitution.

In yet another embodiment, the modified IL-2 polypeptide has at least about 80% sequence identity, e.g., at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity in the regions of amino acid residues 10-25 and 80-100 to the corresponding regions of a comparable IL-2 polypeptide comprising an amino acid sequence set forth in SEQ ID NO:1 or SEQ ID NO:2 without the substitution.

In yet another embodiment, the modified IL-2 polypeptide has at least about 80% sequence identity, e.g., at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity in the regions of amino acid residues 10-25 and 100-134 to the corresponding regions of a comparable IL-2 polypeptide comprising an amino acid sequence set forth in SEQ ID NO:1 or SEQ ID NO:2 without the substitution.

In yet another embodiment, the modified IL-2 polypeptide has at least about 80% sequence identity, e.g., at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity in the regions of amino acid residues 80-100 and 100-134 to the corresponding regions of a comparable IL-2 polypeptide comprising an amino acid sequence set forth in SEQ ID NO:1 or SEQ ID NO:2 without the substitution.

In yet another embodiment, the modified IL-2 polypeptide has at least about 80% sequence identity, e.g., at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity in the regions of amino acid residues 10-25, 80-100 and 100-134 to the corresponding regions of a comparable IL-2 polypeptide comprising an amino acid sequence set forth in SEQ ID NO:1 or SEQ ID NO:2 without the substitution.

In one embodiment, the modified IL-2 polypeptide has at least about 50% sequence identity, e.g., at least about 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99% sequence identity or more to a comparable IL-2 polypeptide comprising an amino acid sequence set forth in SEQ ID NO:1 or SEQ ID NO:2 without the substitution.

The present modified IL-2 polypeptide can comprise any suitable substitution with a natural amino acid. For example, the present modified IL-2 polypeptide can comprise a substitution with lysine, cysteine, histidine, arginine, aspartic acid, glutamic acid, serine, threonine, alanine, tryptophan, isoleucine, phenylalanine, or tyrosine at a position of Q13, L19, N29, N30, Y31, K32, N33, P34, K35, T37, R38, T41, F42, K43, Y45, K48, K49, E62, K64, P65, N71, Q74, K76, R81, L85, S87, V91, I92, V93 or a combination thereof.

In one embodiment, the present modified IL-2 polypeptide: a) comprises a substitution with a natural amino acid at a position selected from the group consisting of N29, N30, Y31, K32, N33, P34, K35, R38, T41, F42, K43, Y45, K48, K49, E62, K64, P65, N71, Q74, K76 and a combination thereof, and is configured to be conjugated to a water-soluble polymer, a lipid, a protein, or a peptide at the position selected from the group consisting of N29, N30, Y31, K32, N33, P34, K35, R38, T41, F42, K43, Y45, K48, K49, E62, K64, P65, N71, Q74, K76 and a combination thereof; and/or b) comprises a substitution with a natural amino acid at a position selected from the group consisting of N29, N30, Y31, K32, N33, P34, K35, R38, T41, F42, K43, Y45, K48, K49, E62, K64, P65, N71, Q74, K76 and a combination thereof, and is configured to be conjugated to a water-soluble polymer, a lipid, a protein, or a peptide at the N terminal and/or C terminal of the polypeptide.

In another embodiment, the present modified IL-2 polypeptide: a) comprises a substitution with lysine, cysteine, histidine, arginine, aspartic acid, glutamic acid, serine, threonine, alanine, tryptophan, isoleucine, phenylalanine, or tyrosine at a position selected from the group consisting of N29, N30, Y31, K32, N33, P34, K35, R38, T41, F42, K43, Y45, K48, K49, E62, K64, P65, N71, Q74, K76 and a combination thereof; and/or b) comprises a substitution with lysine, cysteine, histidine, arginine, aspartic acid, glutamic acid, serine, threonine, alanine, tryptophan, isoleucine, phenylalanine, or tyrosine at a position selected from the group consisting of N29, N30, Y31, N33, P34, K35, R38, T41, K43, K48, K49, K64, P65, N71, Q74, K76 and a combination thereof.

In still another embodiment, the present modified IL-2 polypeptide: a) comprises a substitution with cysteine at a position selected from the group consisting of N29, N30, Y31, N33, P34, K35, R38, T41, K43, K48, K49, K64, P65, N71, Q74, K76 and a combination thereof; b) comprises a substitution with cysteine at a position selected from the group consisting of N29, Y31, K35, P65, N71, Q74 and a combination thereof; c) comprises a substitution with cysteine at a position of Y31; and/or d) comprises a substitution with cysteine at a position of P65.

In still another embodiment, the present modified IL-2 polypeptide comprises a substitution with any amino acid at a position Y31. For example, the present modified IL-2 polypeptide can comprise a substitution with serine or alanine at a position Y31.

The present modified IL-2 polypeptide can further comprise a substitution with a natural amino acid or an unnatural amino acid at a position within IL-2Rα interaction region, IL-2Rβ interaction region and/or IL-2Rγ interaction region.

The present modified IL-2 polypeptide can further comprise a substitution with a natural amino acid at a position within IL-2Rα interaction region. The present modified IL-2 polypeptide can further comprise a substitution with a natural amino acid at any suitable position within IL-2Rα interaction region. For example, the present modified IL-2 polypeptide can further comprise a substitution with a natural amino acid at a position selected from the group consisting of R38, F42, Y45, E62, P65 and a combination thereof.

The present modified IL-2 polypeptide can comprise any suitable substitution with a natural amino acid at a position within IL-2Rα interaction region. For example, the present modified IL-2 polypeptide can comprise a substitution with lysine, cysteine, histidine, arginine, aspartic acid, glutamic acid, serine, threonine, alanine, tryptophan, isoleucine, phenylalanine, or tyrosine at a position selected from the group consisting of R38, F42, Y45, E62, P65 and a combination thereof.

In one embodiment, the present modified IL-2 polypeptide: a) comprises a substitution with cysteine at a position selected from the group consisting of R38, F42, Y45, E62, P65 and a combination thereof; b) comprises a substitution with alanine, lysine or serine at a position of F42; c) comprises a substitution with alanine at a position of F42; d) comprises a substitution with serine at a position of F42; e) comprises a substitution with lysine at a position of F42; f) comprises a substitution with alanine, histidine or serine at a position of Y45; g) comprises a substitution with alanine at a position of Y45; h) comprises a substitution with histidine at a position of Y45; i) comprises a substitution with alanine, aspartic acid or serine at a position of R38; j) comprises a substitution with aspartic acid at a position of R38; k) comprises a substitution with alanine at a position of P65; l) comprises a substitution with serine at a position of P65; m) comprises a substitution with alanine at a position of E62; and/or n) comprises a substitution with lysine at a position of F42, a substitution with cysteine at position of Y31, or a combination thereof.

The present modified IL-2 polypeptide can further comprise a substitution with a natural amino acid at a position within IL-2Rβ interaction region. The present modified IL-2 polypeptide can further comprise a substitution with a natural amino acid at any suitable position within IL-2Rβ interaction region. For example, the present modified IL-2 polypeptide can further comprise a substitution with a natural amino acid at a position selected from the group consisting of Q13, L19, R81, L85, S87, V91, I92, V93 and a combination thereof.

The present modified IL-2 polypeptide can comprise any suitable substitution with a natural amino acid at a position within IL-2Rβ interaction region. For example, the present modified IL-2 polypeptide can comprise a substitution with lysine, cysteine, histidine, arginine, aspartic acid, glutamic acid, serine, threonine, alanine, tryptophan, isoleucine, phenylalanine, or tyrosine at a position selected from the group consisting of Q13, L19, R81, L85, S87, V91, I92, V93 and a combination thereof. In one embodiment, the present modified IL-2 polypeptide can comprise a substitution with cysteine at a position selected from the group consisting of Q13, L19, R81, L85, S87, V91, I92, V93 and a combination thereof.

In one embodiment, the present modified IL-2 polypeptide can further comprise: a) a substitution with a natural amino acid at a position within IL-2Rα interaction region and a substitution with a natural amino acid at a position within IL-2Rβ interaction region; b) a substitution with a natural amino acid at a position within IL-2Rα interaction region and a substitution with a natural amino acid at a position within IL-2Rγ interaction region; or c) a substitution with a natural amino acid at a position within IL-2Rα interaction region, a substitution with a natural amino acid at a position within IL-2Rβ interaction region and a substitution with a natural amino acid at a position within IL-2Rγ interaction region.

The present modified IL-2 polypeptide can comprise any suitable substitution with an unnatural amino acid. For example, the unnatural amino acids disclosed in WO 2019/028425 A1 and WO 2019/028419 A1 can be used. In one embodiment, an unnatural amino acid can be a lysine analogue, a cysteine analogue or a histidine analogue, comprises an aromatic side chain; comprises an azido group; comprises an alkyne group; or comprises an aldehyde or ketone group. In another embodiment, the unnatural amino acid does not comprise an aromatic side chain. In still another embodiment, the unnatural amino acid comprises N6-azidoethoxy-L-lysine (AzK), N6-propargylethoxy-L-lysine (PraK), BCN-L-lysine, norbornene lysine, TCO-lysine, methyltetrazine lysine, allyloxycarbonyllysine, 2-amino-8-oxononanoic acid, 2-amino-8-oxooctanoic acid, p-acetyl-L-phenylalanine, p-azidomethyl-L-phenylalanine (pAMF), p-iodo-L-phenylalanine, m-acetylphenylalanine, 2-amino-8-oxononanoic acid, p-propargyloxyphenylalanine, p-propargyl-phenylalanine, 3-methyl-phenylalanine, L-Dopa, fluorinated phenylalanine, isopropyl-L-phenylalanine, p-azido-L-phenylalanine, p-acyl-L-phenylalanine, p-benzoyl-L-phenylalanine, p-bromophenylalanine, p-amino-L-phenylalanine, isopropyl-L-phenylalanine, O-allyltyrosine, O-methyl-L-tyrosine, 0-4-allyl-L-tyrosine, 4-propyl-L-tyrosine, phosphonotyrosine, tri-O-acetyl-GlcNAcp-serine, L-phosphoserine, phosphonoserine, L-3-(2-naphthyl)alanine, 2-amino-3-((2-((3-(benzyloxy)-3-oxopropyl)amino)ethyl)selanyl)propanoic acid, 2-amino-3-(phenylselanyl)propanoic, or selenocysteine.

The unnatural amino acid can be incorporated into the modified IL-2 polypeptide by any suitable means or methods. For example, the unnatural amino acid can be incorporated into the modified IL-2 polypeptide by an orthogonal tRNA synthetase/tRNA pair. Any suitable orthogonal tRNA can be used. For example, the orthogonal tRNA of the orthogonal synthetase/tRNA pair can comprise at least one unnatural nucleobase.

The present modified IL-2 polypeptide can have reduced or no detectable binding to an IL-2Rα compared to a comparable IL-2 polypeptide comprising an amino acid sequence set forth in SEQ ID NO:1 or SEQ ID NO:2 without the substitution. In one embodiment, the binding affinity of the present modified IL-2 polypeptide to an IL-2Rα can be decreased from about 10% to about 100%, e.g., decreased by about 10%, 20%, 30%, 40%, 50%, 60%, 17%, 80%, 90%, 100%, or a subrange thereof. In another embodiment, the binding affinity of the present modified IL-2 polypeptide to an IL-2Rα can be decreased from about 10% to about 100%, or can be decreased from about 1 fold to about 100,000 fold or more, e.g., decreased by about 1 fold, 10 fold, 100 fold, 1,000 fold, 10,000 fold, 100,000 fold or more, or a subrange thereof. In still another embodiment, the present modified IL-2 polypeptide has no detectable binding to an IL-2Rα.

The present modified IL-2 polypeptide can have reduced or no detectable receptor signaling potency to IL-2Rαβγ compared to a comparable IL-2 polypeptide comprising an amino acid sequence set forth in SEQ ID NO:1 or SEQ ID NO:2 without the substitution. In one embodiment, a ratio between the signaling potency to IL-2Rαβγ of the present modified IL-2 polypeptide and the signaling potency to IL-2Rαβγ of the comparable IL-2 polypeptide comprising an amino acid sequence set forth in SEQ ID NO:1 or SEQ ID NO:2 without the substitution can be from about ½ to about 1/100,000, e.g., at about ½, ⅕, 1/10, 1/100, 1/1,000, 1/10,000, 1/100,000, or More, or a Subrange Thereof. In Another embodiment, the present modified IL-2 polypeptide has no detectable receptor signaling potency to IL-2Rαβγ.

In one embodiment, the present modified IL-2 polypeptide has reduced binding to an IL-2Rα compared to a comparable IL-2 polypeptide comprising an amino acid sequence set forth in SEQ ID NO:1 or SEQ ID NO:2 without the substitution and has reduced receptor signaling potency to IL-2Rαβγ compared to a comparable IL-2 polypeptide comprising an amino acid sequence set forth in SEQ ID NO:1 or SEQ ID NO:2 without the substitution. In another embodiment, the present modified IL-2 polypeptide has no detectable binding to an IL-2Rα and has no detectable receptor signaling potency to IL-2Rαβγ.

The present modified IL-2 polypeptide can retain substantial or can have higher binding level to an interleukin 2 receptor (3 (IL-2Rβ) or an interleukin 2 receptor γ (IL-2Rγ) compared to a comparable IL-2 polypeptide comprising an amino acid sequence set forth in SEQ ID NO:1 or SEQ ID NO:2 without the substitution, and/or can retain substantial or can have higher receptor signaling potency to IL-2Rβγ compared to a comparable IL-2 polypeptide comprising an amino acid sequence set forth in SEQ ID NO:1 or SEQ ID NO:2 without the substitution. In one embodiment, the present modified IL-2 polypeptide retains substantial or has higher binding level to an IL-2Rβ or an IL-2Rγ compared to a comparable IL-2 polypeptide comprising an amino acid sequence set forth in SEQ ID NO:1 or SEQ ID NO:2 without the substitution. In another embodiment, the present modified IL-2 polypeptide retains substantial or has higher receptor signaling potency to IL-2Rβγ compared to a comparable IL-2 polypeptide comprising an amino acid sequence set forth in SEQ ID NO:1 or SEQ ID NO:2 without the substitution. In still another embodiment, the present modified IL-2 polypeptide retains substantial or has higher binding level to an IL-2Rβ or an IL-2Rγ compared to a comparable IL-2 polypeptide comprising an amino acid sequence set forth in SEQ ID NO:1 or SEQ ID NO:2 without the substitution, and retains substantial or has higher receptor signaling potency to IL-2Rβγ compared to a comparable IL-2 polypeptide comprising an amino acid sequence set forth in SEQ ID NO:1 or SEQ ID NO:2 without the substitution.

The present modified IL-2 polypeptide can comprise a deletion at any suitable location. In one embodiment, the present modified IL-2 polypeptide has a N terminal deletion, e.g., a N terminal deletion of amino acid residues 1-30 or a subrange thereof. In another embodiment, the present modified IL-2 polypeptide has a C terminal deletion, e.g., a C terminal deletion of amino acid residues 114-134 or a subrange thereof. In still another embodiment, the present modified IL-2 polypeptide has a N terminal deletion and a C terminal deletion.

The present modified IL-2 polypeptide can be a part of a fusion polypeptide, e.g., a recombinant fusion protein, that comprises the modified IL-2 polypeptide and an additional amino acid sequence. The present modified IL-2 polypeptide can be fused to the additional amino acid sequence in any suitable manner. For example, the N terminus or the C terminus of the modified IL-2 polypeptide can be fused to the additional amino acid sequence. The additional amino acid sequence can comprise any suitable sequence or content. For example, the additional amino acid sequence can comprise an antibody sequence or a portion or a fragment thereof. In another example, the additional amino acid sequence can comprise a Fc portion of an antibody.

The present modified IL-2 polypeptide can be in any suitable form. For example, the present modified IL-2 polypeptide can be in an isolated or purified form.

The present modified IL-2 polypeptide can be prepare using any suitable technique or process. For example, The present modified IL-2 polypeptide can be prepare by recombinant production, chemical synthesis or a combination thereof.

In another aspect, the present invention is directed to a polynucleotide, e.g., DNA, RNA or viral vector, that encodes a modified IL-2 polypeptide as described above and is configured to express said modified IL-2 polypeptide in vitro and/or in vivo.

The present modified IL-2 polypeptide can be applied in any suitable form. For example, the modified IL-2 polypeptide, as described above, with or without conjugate, can be applied in the format of protein, fusion protein, protein conjugate, or as part of nanoparticles. In some embodiments, a polynucleotide, e.g., DNA, RNA or viral vector, that encodes the modified IL-2 polypeptide and is configured to express said modified IL-2 polypeptide in vitro and/or in vivo, can be applied to cell(s), tissue(s), organ(s), or subject(s), e.g., human subject(s).

### D. Modified Interleukin 2 (IL-2) Polypeptide Conjugates

In another aspect, the present invention is directed to modified IL-2 polypeptide conjugate, which comprises a modified IL-2 polypeptide, as described above, that is conjugated to another moiety, e.g., a water-soluble polymer, a lipid, a polypeptide, e.g., a protein, or a peptide.

The modified IL-2 polypeptide can be conjugated to another moiety, e.g., a water-soluble polymer, a lipid, a protein, or a peptide, in any suitable manner. For example, the modified IL-2 polypeptide can be conjugated to a water-soluble polymer, a lipid, a protein, or a peptide covalently. In another example, the modified IL-2 polypeptide can be conjugated to a water-soluble polymer, a lipid, a protein, or a peptide non-covalently. In still another example, the modified IL-2 polypeptide can be conjugated to a water-soluble polymer, a lipid, a protein, or a peptide via a substituted natural amino acid or unnatural amino acid at any suitable position.

In one embodiment, the modified IL-2 polypeptide is conjugated to another moiety, e.g., a water-soluble polymer, a lipid, a protein, or a peptide, via a substituted natural amino acid or unnatural amino acid at a position selected from the group consisting of Q13, L19, N29, N30, Y31, K32, N33, P34, K35, T37, R38, T41, F42, K43, Y45, K48, K49, E62, K64, P65, N71, Q74, K76, R81, L85, S87, V91, I92, V93 and a combination thereof. In another embodiment, the modified IL-2 polypeptide is conjugated to another moiety, e.g., a water-soluble polymer, a lipid, a protein, or a peptide, via a substituted natural amino acid at a position selected from the group consisting of Q13, L19, N29, N30, Y31, K32, N33, P34, K35, T37, R38, T41, F42, K43, Y45, K48, K49, E62, K64, P65, N71, Q74, K76, R81, L85, S87, V91, I92, V93 and a combination thereof. In still another embodiment, the modified IL-2 polypeptide is conjugated to another moiety, e.g., a water-soluble polymer, a lipid, a protein, or a peptide, via a substituted lysine, cysteine, histidine, arginine, aspartic acid, glutamic acid, serine, threonine, alanine, tryptophan, isoleucine, phenylalanine, or tyrosine at a position selected from the group consisting of Q13, L19, N29, N30, Y31, K32, N33, P34, K35, T37, R38, T41, F42, K43, Y45, K48, K49, E62, K64, P65, N71, Q74, K76, R81, L85, S87, V91, I92, V93 and a combination thereof. In yet another embodiment, the modified IL-2 polypeptide is conjugated to another moiety, e.g., a water-soluble polymer, a lipid, a protein, or a peptide, via a substituted cysteine at a position selected from the group consisting of Q13, L19, N29, N30, Y31, K32, N33, P34, K35, T37, R38, T41, F42, K43, Y45, K48, K49, E62, K64, P65, N71, Q74, K76, R81, L85, S87, V91, I92, V93 and a combination thereof.

The modified IL-2 polypeptide can be conjugated to another moiety, e.g., a water-soluble polymer, a lipid, a protein, or a peptide, via a substituted natural amino acid or unnatural amino acid at a position selected from the group consisting of N29, N30, Y31, K32, N33, P34, K35, R38, T41, F42, K43, Y45, K48, K49, E62, K64, P65, N71, Q74, K76 and a combination thereof. In one embodiment, the modified IL-2 polypeptide is conjugated to another moiety, e.g., a water-soluble polymer, a lipid, a protein, or a peptide, via a substituted natural amino acid at a position selected from the group consisting of N29, N30, Y31, K32, N33, P34, K35, R38, T41, F42, K43, Y45, K48, K49, E62, K64, P65, N71, Q74, K76 and a combination thereof. In another embodiment, the modified IL-2 polypeptide is conjugated to another moiety, e.g., a water-soluble polymer, a lipid, a protein, or a peptide, via a substituted lysine, cysteine, histidine, arginine, aspartic acid, glutamic acid, serine, threonine, alanine, tryptophan, isoleucine, phenylalanine, or tyrosine at a position selected from the group consisting of N29, N30, Y31, K32, N33, P34, K35, R38, T41, F42, K43, Y45, K48, K49, E62, K64, P65, N71, Q74, K76 and a combination thereof. In still another embodiment, the modified IL-2 polypeptide is conjugated to another moiety, e.g., a water-soluble polymer, a lipid, a protein, or a peptide, via a substituted cysteine at a position selected from the group consisting of N29, N30, Y31, K32, N33, P34, K35, R38, T41, F42, K43, Y45, K48, K49, E62, K64, P65, N71, Q74, K76 and a combination thereof.

The the modified IL-2 polypeptide can be conjugated to another moiety, e.g., a water-soluble polymer, a lipid, a protein, or a peptide, via a single amino acid residue or multiple amino acid residues of the modified IL-2 polypeptide. In one embodiment, the modified IL-2 polypeptide can be conjugated to another moiety, e.g., a water-soluble polymer, a lipid, a protein, or a peptide, via: i) the alpha amino group of the N-terminal amino acid residue of the modified IL-2 polypeptide; ii) the epsilon amino group of a lysine amino acid residue of the modified IL-2 polypeptide; or iii) an N-glycosylation site or 0-glycosylation site of the modified IL-2 polypeptide.

The modified IL-2 polypeptide can be covalently conjugated to another moiety, e.g., a water-soluble polymer, a lipid, a protein, or a peptide, through a linker. The modified IL-2 polypeptide can also be covalently conjugated to another moiety, e.g., a water-soluble polymer, a lipid, a protein, or a peptide, directly without a linker.

The modified IL-2 polypeptide can be conjugated to another moiety, e.g., a water-soluble polymer, a lipid, a protein, or a peptide, via a single amino acid residue in a fusion polypeptide that comprises the modified IL-2 polypeptide and an additional amino acid sequence. The single amino acid residue can be located at any suitable location. For example, the single amino acid residue can be located within the modified IL-2 polypeptide. In another example, the single amino acid residue can be located within the additional amino acid sequence.

The additional amino acid sequence in the present modified IL-2 polypeptide conjugate can comprise any suitable sequence or content. For example, the additional amino acid sequence in the present modified IL-2 polypeptide conjugate can comprise an antibody sequence or a portion or a fragment thereof. In another example, the additional amino acid sequence in the present modified IL-2 polypeptide conjugate can comprise a Fc portion of an antibody.

The modified IL-2 polypeptide can be conjugated to another moiety, e.g., a water-soluble polymer, a lipid, a protein, or a peptide in a fusion polypeptide, in any suitable manner. For example, the modified IL-2 polypeptide can be conjugated to another moiety, e.g., a water-soluble polymer, a lipid, a protein, or a peptide, via: i) the alpha amino group of the N-terminal amino acid residue of the fusion polypeptide; ii) the epsilon amino group of a lysine amino acid residue of the fusion polypeptide; or iii) an N-glycosylation site or O-glycosylation site of the fusion polypeptide. In another example, the fusion polypeptide can be covalently conjugated to a water-soluble polymer, a lipid, a protein, or a peptide directly or through a linker.

The present modified IL-2 polypeptide can be conjugated to any suitable water-soluble polymer. For example, the water-soluble polymer can comprise polyethylene glycol (PEG), poly(propylene glycol) (PPG), copolymers of ethylene glycol and propylene glycol, poly(oxyethylated polyol), poly(olefinic alcohol), poly(vinylpyrrolidone), poly(hydroxyalkylmethacrylamide), poly(hydroxyalkylmethacrylate), poly(saccharides), poly(a-hydroxy acid), poly(vinyl alcohol), polyphosphazene, polyoxazolines (POZ), poly(N-acryloylmorpholine), or a combination thereof. See e.g., WO 2019/028425A1 and WO 2019/028419A1.

In the present modified IL-2 polypeptide conjugate, the water-soluble polymer can comprise a PEG molecule. The PEG molecule can be a linear PEG or a branched PEG. The branched PEG can have any suitable configuration and/or any suitable number of PEG chains. For example, the branched PEG can have about three to about ten PEG chains emanating from a central core group. In another example, the branched PEG can be a star PEG comprising from about 10 to about 100 PEG chains emanating from a central core group. In still another example, the branched PEG can be a comb PEGs comprising multiple PEG chains grafted onto a polymer backbone.

The PEG molecule in the present modified IL-2 polypeptide conjugate can have any suitable molecular weight. For example, the PEG molecule can have a range of molecular weight from about 300 g/mol to about 10,000,000 g/mol, e.g., at about 300 g/mol, 500 g/mol, 1,000 g/mol, 10,000 g/mol, 100,000 g/mol, 1,000,000 g/mol, 10,000,000 g/mol or a subrange thereof. In another example, the PEG molecule can have an average molecular weight from about 5,000 Daltons to about 1,000,000 Daltons, e.g., at about 5,000 Daltons, 10,000 Daltons, 100,000 Daltons, 1,000,000 Daltons or a subrange thereof. In still another example, the PEG molecule can have an average molecular weight of from about 20,000 Daltons to about 30,000 Daltons, e.g., at about 20,000 Daltons, 21,000 Daltons, 22,000 Daltons, 23,000 Daltons, 24,000 Daltons, 25,000 Daltons, 26,000 Daltons, 27,000 Daltons, 28,000 Daltons, 29,000 Daltons, 30,000 Daltons or a subrange thereof.

The PEG molecule in the present modified IL-2 polypeptide conjugate can be in any suitable form. For example, the PEG molecule can be a monodisperse, uniform, or discrete PEG molecule.

The water-soluble polymer in the present modified IL-2 polypeptide conjugate can comprise a polysaccharide.

The modified IL-2 polypeptide in the present modified IL-2 polypeptide conjugate can be conjugated to any suitable lipid. For example, the lipid in the present modified IL-2 polypeptide conjugate can comprise a fatty acid.

The modified IL-2 polypeptide in the present modified IL-2 polypeptide conjugate can be conjugated to any suitable protein. For example, the protein in the present modified IL-2 polypeptide conjugate can comprise an antibody or a binding fragment thereof. The antibody or a binding fragment thereof can comprise an Fc portion of an antibody.

In the present modified IL-2 polypeptide conjugate, the other moiety, e.g., a water-soluble polymer, a lipid, a protein, or a peptide, can be bound to the modified IL-2 polypeptide via any suitable manner. For example, the other moiety, e.g., a water-soluble polymer, a lipid, a protein, or a peptide, can be indirectly bound to the substituted natural amino acid or unnatural amino acid of the modified IL-2 polypeptide through a linker. In another example, the other moiety, e.g., a water-soluble polymer, a lipid, a protein, or a peptide, can be directly bound to the substituted natural amino acid or unnatural amino acid of the modified IL-2 polypeptide.

The present modified IL-2 polypeptide conjugate can have any suitable half-life in vivo. For example, the present modified IL-2 polypeptide conjugate can have a half-life in vivo from about 5 minutes to about 10 days, e.g., at about 5 minutes, 10 minutes, 20 minutes, 30 minutes, 40 minutes, 50 minutes, 1 hours, 2 hours, 3 hours, 4 hours, 5 hours, 6 hours, 7 hours, 8 hours, 9 hours, 10 hours, 11 hour, 12 hours, 13 hours, 14 hours, 15 hours, 16 hours, 17 hours, 18 hours, 19 hours, 20 hours, 21 hour, 22 hours, 23 hours, 1 day, 2 days, 3 days, 4 days, 5 days, 6 days, 7 days, 8 days, 9 days, 10 days or a subrange thereof.

### E. Pharmaceutical Compositions

In another aspect, the present invention is directed to a pharmaceutical composition comprising an effective amount of a modified IL-2 polypeptide, a polynucleotide, e.g., DNA, RNA or viral vector, or a modified IL-2 polypeptide conjugate, as described above, and a pharmaceutically acceptable carrier or excipient.

The present pharmaceutical composition can be configured to treat or prevent any suitable disease(s), disorder(s) or condition(s). For example, the present pharmaceutical composition can be configured to treat or prevent a proliferation disorder in a subject.

In one embodiment, the present pharmaceutical composition is configured to treat or prevent a solid tumor or cancer in a subject. The solid tumor or cancer can be Chondrosarcoma, Ewing's sarcoma, Malignant fibrous histiocytoma of bone/osteosarcoma, Osteosarcoma, Rhabdomyosarcoma, Heart cancer, Astrocytoma, Brainstem glioma, Pilocytic astrocytoma, Ependymoma, Primitive neuroectodermal tumor, Cerebellar astrocytoma, Cerebral astrocytoma, Glioma, Medulloblastoma, Neuroblastoma, Oligodendroglioma, Pineal astrocytoma, Pituitary adenoma, Visual pathway and hypothalamic glioma, Breast cancer, Invasive lobular carcinoma, Tubular carcinoma, Invasive cribriform carcinoma, Medullary carcinoma, Male breast cancer, Phyllodes tumor, Inflammatory Breast Cancer, Adrenocortical carcinoma, Islet cell carcinoma (endocrine pancreas), Multiple endocrine neoplasia syndrome, Parathyroid cancer, Pheochromocytoma, Thyroid cancer, Merkel cell carcinoma, Uveal melanoma, Retinoblastoma, Anal cancer, Appendix cancer, cholangiocarcinoma, Carcinoid tumor, gastrointestinal, Colon cancer, Extrahepatic bile duct cancer, Gallbladder cancer, Gastric (stomach) cancer, Gastrointestinal carcinoid tumor, Gastrointestinal stromal tumor (GIST), Hepatocellular cancer, Pancreatic cancer islet cell, Rectal cancer, Bladder cancer, Cervical cancer, Endometrial cancer, Extragonadal germ cell tumor, Ovarian cancer, Ovarian epithelial cancer (surface epithelial-stromal tumor), Ovarian germ cell tumor, Penile cancer, Renal cell carcinoma, Renal pelvis and ureter, transitional cell cancer, Prostate cancer, Testicular cancer, Gestational trophoblastic tumor, Ureter and renal pelvis, transitional cell cancer, Urethral cancer, Uterine sarcoma, Vaginal cancer, Vulvar cancer, Wilms tumor, Esophageal cancer, Head and neck cancer, Nasopharyngeal carcinoma, Oral cancer, Oropharyngeal cancer, Paranasal sinus and nasal cavity cancer, Pharyngeal cancer, Salivary gland cancer, Hypopharyngeal cancer, Basal-cell carcinoma, Melanoma, Skin cancer (non-melanoma), Bronchial adenomas/carcinoids, Small cell lung cancer, Mesothelioma, Non-small cell lung cancer, Pleuropulmonary blastoma, Laryngeal cancer, Thymoma and thymic carcinoma, AIDS-related cancers, Kaposi sarcoma, Epithelioid hemangioendothelioma (EHE), Desmoplastic small round cell tumor or Liposarcoma.

In another embodiment, the present pharmaceutical composition is configured to treat or prevent a hematological malignancy in a subject. The hematological malignancy can be hematological malignancy including: myeloid neoplasms, Leukemias, Lymphomas, Hodgkin lymphoma, Non-Hodgkin lymphoma, Anaplastic large cell lymphoma, Angioimmunoblastic T-cell lymphoma, Hepatosplenic T-cell lymphoma, B-cell lymphoma reticuloendotheliosis, Reticulosis, Microglioma, Diffuse large B-cell lymphoma, Follicular lymphoma, Mucosa-associated lymphatic tissue lymphoma, B-cell chronic lymphocytic leukemia, Mantle cell lymphoma, Burkitt lymphoma, Mediastinal large B cell lymphoma, Waldenström's macroglobulinemia, Nodal marginal zone B cell lymphoma, Splenic marginal zone lymphoma, Intravascular large B-cell lymphoma, Primary effusion lymphoma, Lymphomatoid granulomatosis, Nodular lymphocyte predominant Hodgkin's lymphoma, plasma cell leukemia, Acute erythraemia and erythroleukaemia, Acute erythremic myelosis, Acute erythroid leukemia, Heilmeyer-Schoner disease, Acute megakaryoblastic leukemia, Mast cell leukemia, Panmyelosis, Acute panmyelosis with myelofibrosis, Lymphosarcoma cell leukemia, Acute leukaemia of unspecified cell type, Blastic phase chronic myelogenous leukemia, Stem cell leukemia, Chronic leukaemia of unspecified cell type, Subacute leukaemia of unspecified cell type, Accelerated phase chronic myelogenous leukemia, Acute myeloid leukemia, Polycythemia vera, Acute promyelocytic leukemia, Acute basophilic leukemia, Acute eosinophilic leukemia, Acute lymphoblastic leukemia, Acute monocytic leukemia, Acute myeloblastic leukemia with maturation, Acute myeloid dendritic cell leukemia, Adult T-cell leukemia/lymphoma, Aggressive NK-cell leukemia, B-cell prolymphocytic leukemia, B-cell chronic lymphocytic leukemia, B-cell leukemia, Chronic myelogenous leukemia, Chronic myelomonocytic leukemia, Chronic neutrophilic leukemia, Chronic lymphocytic leukemia, Hairy cell leukemia, Chronic idiopathic myelofibrosis, Multiple myeloma, Kahler's disease, Myelomatosis, Solitary myeloma, Plasma cell leukemia, Plasmacytoma, extramedullary, Malignant plasma cell tumour NOS, Plasmacytoma NOS, Monoclonal gammopathy, Multiple Myeloma, Angiocentric immunoproliferative lesion, Lymphoid granulomatosis, Angioimmunoblastic lymphadenopathy, T-gamma lymphoproliferative disease, Waldenström's macroglobulinaemia, Alpha heavy chain disease, Gamma heavy chain disease, Franklin's disease, Immunoproliferative small intestinal disease, Mediterranean disease, Malignant immunoproliferative disease, unspecified, or Immunoproliferative disease NOS.

In still another embodiment, the present pharmaceutical composition is configured to treat or prevent an immune deficiency disease or disorder in a subject. The immune deficiency disease or disorder can be Agammaglobulinemia: X-Linked and Autosomal Recessive, Ataxia Telangiectasia, Chronic Granulomatous Disease and Other Phagocytic Cell Disorders, Common Variable Immune Deficiency, Complement Deficiencies, DiGeorge Syndrome, Hemophagocytic Lymphohistiocytosis (HLH), Hyper IgE Syndrome, Hyper IgM Syndromes, IgG Subclass Deficiency, Innate Immune Defects, NEMO Deficiency Syndrome, Selective IgA Deficiency, Selective IgM Deficiency, Severe Combined Immune, Deficiency and Combined Immune Deficiency, Specific Antibody Deficiency, Transient Hypogammaglobulinemia of Infancy, WHIM Syndrome (Warts, Hypogammaglobulinemia, Infections, and Myelokathexis), Wiskott-Aldrich Syndrome, Other Antibody Deficiency Disorders, Other Primary Cellular Immunodeficiencies, Severe combined immune deficiency (SCID), Common variable immune deficiency (CVID), Human immunodeficiency virus/acquired immune deficiency syndrome (HIV/AIDS), Drug-induced immune deficiency, Graft versus host syndrome, Primary Immune Deficiency Diseases (PIDDs) or Lymphopenia.

The present pharmaceutical composition can further comprise another active ingredient. The another active ingredient can the active ingredient to treat or prevent any suitable any suitable disease(s), disorder(s) or condition(s). For example, the another active ingredient can be an anti-neoplasm substance.

The additional active ingredient(s) may be formulated in a separate pharmaceutical composition from at least one exemplary modified IL-2 polypeptide or modified IL-2 polypeptide conjugate of the present disclosure or may be included with at least one exemplary modified IL-2 polypeptide or modified IL-2 polypeptide conjugate of the present disclosure in a single pharmaceutical composition.

The present pharmaceutical compositions can be formulated to be administered orally, parenterally, by inhalation, topically, rectally, nasally, buccally, vaginally, via an implanted reservoir, or other drug administration methods. The term “parenteral” as used herein includes subcutaneous, intracutaneous, intravenous, intramuscular, intraarticular, intraarterial, intrasynovial, intrasternal, intrathecal, intralesional and intracranial injection or infusion techniques.

A sterile injectable composition, such as a sterile injectable aqueous or oleaginous suspension, may be formulated according to techniques known in the art using suitable dispersing or wetting agents and suspending agents. The sterile injectable preparation may also be a sterile injectable solution or suspension in a non-toxic parenterally acceptable diluent or solvent. Among the acceptable vehicles and solvents that may be employed include mannitol, water, Ringer's solution and isotonic sodium chloride solution. Suitable carriers and other pharmaceutical composition components are typically sterile.

In addition, sterile, fixed oils are conventionally employed as a solvent or suspending medium (e.g., synthetic mono- or diglycerides). Fatty acids, such as oleic acid and its glyceride derivatives, are useful in the preparation of injectables, as are pharmaceutically acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Various emulsifying agents or bioavailability enhancers which are commonly used in the manufacture of pharmaceutically acceptable solid, liquid, or other dosage forms can also be used for the purpose of formulation.

A composition for oral administration may be any orally acceptable dosage form including, but not limited to, tablets, capsules, emulsions and aqueous suspensions, dispersions and solutions. In the case of tablets for oral use, commonly used carriers include lactose and corn starch. Lubricating agents, such as magnesium stearate, can also be added. For oral administration in a capsule form, useful diluents include lactose and dried corn starch. When aqueous suspensions or emulsions are administered orally, the active ingredient can be suspended or dissolved in an oily phase combined with emulsifying or suspending agents. If needed, certain sweetening, flavoring, or coloring agents can be added. A nasal aerosol or inhalation compositions can be prepared according to techniques well-known in the art of pharmaceutical formulation and can be prepared as solutions in, for example saline, employing suitable preservatives (for example, benzyl alcohol), absorption promoters to enhance bioavailability, and/or other solubilizing or dispersing agents known in the art.

Any suitable formulation of the compounds described herein can be prepared. See generally, Remington's Pharmaceutical Sciences, (2000) Hoover, J. E. editor, 20 th edition, Lippincott Williams and Wilkins Publishing Company, Easton, Pa., pages 780-857. A formulation is selected to be suitable for an appropriate route of administration. In cases where compounds are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts. Pharmaceutically acceptable salts are obtained using standard procedures well known in the art, for example, by a sufficiently basic compound such as an amine with a suitable acid, affording a physiologically acceptable anion. Alkali metal (e.g., sodium, potassium or lithium) or alkaline earth metal (e.g., calcium) salts of carboxylic acids also are made.

Where contemplated compounds or substances are administered in a pharmacological composition, it is contemplated that the compounds or substances can be formulated in admixture with a pharmaceutically acceptable excipient and/or carrier. For example, contemplated compounds or substances can be administered orally as neutral compounds or substances or as pharmaceutically acceptable salts, or intravenously in a physiological saline solution. Conventional buffers such as phosphates, bicarbonates or citrates can be used for this purpose. Of course, one of ordinary skill in the art may modify the formulations within the teachings of the specification to provide numerous formulations for a particular route of administration. In particular, contemplated compounds or substances may be modified to render them more soluble in water or other vehicle, which for example, may be easily accomplished with minor modifications (salt formulation, esterification, etc.) that are well within the ordinary skill in the art. It is also well within the ordinary skill of the art to modify the route of administration and dosage regimen of a particular compound or substance in order to manage the pharmacokinetics of the present compounds or substances, e.g., the present modified IL-2 polypeptide(s) or modified IL-2 polypeptide conjugate(s), for maximum beneficial effect in a patient.

The present modified IL-2 polypeptide or modified IL-2 polypeptide conjugate may be soluble in organic solvents such as chloroform, dichloromethane, ethyl acetate, ethanol, methanol, isopropanol, acetonitrile, glycerol, N,N-dimethylformamide, N,N-dimetheylaceatmide, dimethylsulfoxide, etc. In one embodiment, the present invention provides formulations prepared by mixing the present modified IL-2 polypeptide or modified IL-2 polypeptide conjugate with a pharmaceutically acceptable carrier. In one aspect, the formulation may be prepared using a method comprising: a) dissolving a described compound or substance in a water-soluble organic solvent, a non-ionic solvent, a water-soluble lipid, a cyclodextrin, a vitamin such as tocopherol, a fatty acid, a fatty acid ester, a phospholipid, or a combination thereof, to provide a solution; and b) adding saline or a buffer containing 1-10% carbohydrate solution. In one example, the carbohydrate comprises dextrose. The pharmaceutical compositions obtained using the present methods are stable and useful for animal and clinical applications.

Illustrative examples of water soluble organic solvents for use in the present pharmaceutical compositions include and are not limited to polyethylene glycol (PEG), alcohols, acetonitrile, N-methyl-2-pyrrolidone, N,N-dimethylformamide, N,N-dimethylacetamide, dimethyl sulfoxide, or a combination thereof. Examples of alcohols include but are not limited to methanol, ethanol, isopropanol, glycerol, or propylene glycol.

Illustrative examples of water soluble non-ionic surfactants for use in the present pharmaceutical compositions include and are not limited to CREMOPHOR® EL, polyethylene glycol modified CREMOPHOR® (polyoxyethyleneglyceroltriricinoleat 35), hydrogenated CREMOPHOR® RH40, hydrogenated CREMOPHOR® RH60, PEG-succinate, polysorbate 20, polysorbate 80, SOLUTOL® HS (polyethylene glycol 660 12-hydroxystearate), sorbitan monooleate, poloxamer, LABRAFIL® (ethoxylated persic oil), LABRASOL® (capryl-caproyl macrogol-8-glyceride), GELUCIRE® (glycerol ester), SOFTIGEN® (PEG 6 caprylic glyceride), glycerin, glycol-polysorbate, or a combination thereof.

Illustrative examples of water soluble lipids for use in the present pharmaceutical compositions include but are not limited to vegetable oils, triglycerides, plant oils, or a combination thereof. Examples of lipid oils include but are not limited to castor oil, polyoxyl castor oil, corn oil, olive oil, cottonseed oil, peanut oil, peppermint oil, safflower oil, sesame oil, soybean oil, hydrogenated vegetable oil, hydrogenated soybean oil, a triglyceride of coconut oil, palm seed oil, and hydrogenated forms thereof, or a combination thereof.

Illustrative examples of fatty acids and fatty acid esters for use in the present pharmaceutical compositions include but are not limited to oleic acid, monoglycerides, diglycerides, a mono- or di-fatty acid ester of PEG, or a combination thereof.

Illustrative examples of cyclodextrins for use in the present pharmaceutical compositions include but are not limited to alpha-cyclodextrin, beta-cyclodextrin, hydroxypropyl-beta-cyclodextrin, or sulfobutyl ether-beta-cyclodextrin.

Illustrative examples of phospholipids for use in the present pharmaceutical compositions include but are not limited to soy phosphatidylcholine, or distearoyl phosphatidylglycerol, and hydrogenated forms thereof, or a combination thereof.

One of ordinary skill in the art may modify the formulations within the teachings of the specification to provide numerous formulations for a particular route of administration. In particular, the compounds or substances may be modified to render them more soluble in water or other vehicle. It is also well within the ordinary skill of the art to modify the route of administration and dosage regimen of a particular compound or substance in order to manage the pharmacokinetics of the present compounds or substances for maximum beneficial effect in a patient.

### F. Methods for Treating or Preventing a Disease or a Disorder

In still another aspect, the present invention is directed to a method for treating or preventing a disease or a disorder, e.g., a proliferation disease or disorder, an autoimmune or inflammatory disease or disorder, or an infectious disease or disorder, in a subject in need comprising administering to said subject an effective amount of a modified IL-2 polypeptide, a polynucleotide, e.g., DNA, RNA or viral vector, a modified IL-2 polypeptide conjugate or a pharmaceutical composition, as described above.

The present method can be used for treating or preventing a disease or a disorder, e.g., a proliferation disease or disorder, in any suitable subject. For example, the present method can be used for treating or preventing a disease or a disorder, e.g., a proliferation disease or disorder, in a human. In another example, the present method can be used for treating or preventing a disease or a disorder, e.g., a proliferation disease or disorder, in a non-human mammal.

In one embodiment, the present method can be used to treat a proliferation disorder in a subject. In another embodiment, the present method can be used to prevent a proliferation disorder in a subject.

The present method can be used for treating or preventing any suitable proliferation disease or disorder in a subject. For example, the present method can be used for treating or preventing a tumor in a subject. In another example, the present method can be used for treating or preventing a cancer in a subject.

In one embodiment, the present method can be used to treat or prevent a solid tumor or cancer in a subject. The present method can be used to treat or prevent any suitable solid tumor or cancer in a subject. For example, the solid tumor or cancer can be Chondrosarcoma, Ewing's sarcoma, Malignant fibrous histiocytoma of bone/osteosarcoma, Osteosarcoma, Rhabdomyosarcoma, Heart cancer, Astrocytoma, Brainstem glioma, Pilocytic astrocytoma, Ependymoma, Primitive neuroectodermal tumor, Cerebellar astrocytoma, Cerebral astrocytoma, Glioma, Medulloblastoma, Neuroblastoma, Oligodendroglioma, Pineal astrocytoma, Pituitary adenoma, Visual pathway and hypothalamic glioma, Breast cancer, Invasive lobular carcinoma, Tubular carcinoma, Invasive cribriform carcinoma, Medullary carcinoma, Male breast cancer, Phyllodes tumor, Inflammatory Breast Cancer, Adrenocortical carcinoma, Islet cell carcinoma (endocrine pancreas), Multiple endocrine neoplasia syndrome, Parathyroid cancer, Pheochromocytoma, Thyroid cancer, Merkel cell carcinoma, Uveal melanoma, Retinoblastoma, Anal cancer, Appendix cancer, cholangiocarcinoma, Carcinoid tumor, gastrointestinal, Colon cancer, Extrahepatic bile duct cancer, Gallbladder cancer, Gastric (stomach) cancer, Gastrointestinal carcinoid tumor, Gastrointestinal stromal tumor (GIST), Hepatocellular cancer, Pancreatic cancer islet cell, Rectal cancer, Bladder cancer, Cervical cancer, Endometrial cancer, Extragonadal germ cell tumor, Ovarian cancer, Ovarian epithelial cancer (surface epithelial-stromal tumor), Ovarian germ cell tumor, Penile cancer, Renal cell carcinoma, Renal pelvis and ureter, transitional cell cancer, Prostate cancer, Testicular cancer, Gestational trophoblastic tumor, Ureter and renal pelvis, transitional cell cancer, Urethral cancer, Uterine sarcoma, Vaginal cancer, Vulvar cancer, Wilms tumor, Esophageal cancer, Head and neck cancer, Nasopharyngeal carcinoma, Oral cancer, Oropharyngeal cancer, Paranasal sinus and nasal cavity cancer, Pharyngeal cancer, Salivary gland cancer, Hypopharyngeal cancer, Basal-cell carcinoma, Melanoma, Skin cancer (non-melanoma), Bronchial adenomas/carcinoids, Small cell lung cancer, Mesothelioma, Non-small cell lung cancer, Pleuropulmonary blastoma, Laryngeal cancer, Thymoma and thymic carcinoma, AIDS-related cancers, Kaposi sarcoma, Epithelioid hemangioendothelioma (EHE), Desmoplastic small round cell tumor or Liposarcoma.

In another embodiment, the present method can be used to treat or prevent a hematological malignancy in a subject. The present method can be used to treat or prevent any suitable hematological malignancy in a subject. For example, the hematological malignancy can be myeloid neoplasms, Leukemias, Lymphomas, Hodgkin lymphoma, Non-Hodgkin lymphoma, Anaplastic large cell lymphoma, Angioimmunoblastic T-cell lymphoma, Hepatosplenic T-cell lymphoma, B-cell lymphoma reticuloendotheliosis, Reticulosis, Microglioma, Diffuse large B-cell lymphoma, Follicular lymphoma, Mucosa-associated lymphatic tissue lymphoma, B-cell chronic lymphocytic leukemia, Mantle cell lymphoma, Burkitt lymphoma, Mediastinal large B cell lymphoma, Waldenström's macroglobulinemia, Nodal marginal zone B cell lymphoma, Splenic marginal zone lymphoma, Intravascular large B-cell lymphoma, Primary effusion lymphoma, Lymphomatoid granulomatosis, Nodular lymphocyte predominant Hodgkin's lymphoma, plasma cell leukemia, Acute erythraemia and erythroleukaemia, Acute erythremic myelosis, Acute erythroid leukemia, Heilmeyer-Schoner disease, Acute megakaryoblastic leukemia, Mast cell leukemia, Panmyelosis, Acute panmyelosis with myelofibrosis, Lymphosarcoma cell leukemia, Acute leukaemia of unspecified cell type, Blastic phase chronic myelogenous leukemia, Stem cell leukemia, Chronic leukaemia of unspecified cell type, Subacute leukaemia of unspecified cell type, Accelerated phase chronic myelogenous leukemia, Acute myeloid leukemia, Polycythemia vera, Acute promyelocytic leukemia, Acute basophilic leukemia, Acute eosinophilic leukemia, Acute lymphoblastic leukemia, Acute monocytic leukemia, Acute myeloblastic leukemia with maturation, Acute myeloid dendritic cell leukemia, Adult T-cell leukemia/lymphoma, Aggressive NK-cell leukemia, B-cell prolymphocytic leukemia, B-cell chronic lymphocytic leukemia, B-cell leukemia, Chronic myelogenous leukemia, Chronic myelomonocytic leukemia, Chronic neutrophilic leukemia, Chronic lymphocytic leukemia, Hairy cell leukemia, Chronic idiopathic myelofibrosis, Multiple myeloma, Kahler's disease, Myelomatosis, Solitary myeloma, Plasma cell leukemia, Plasmacytoma, extramedullary, Malignant plasma cell tumour NOS, Plasmacytoma NOS, Monoclonal gammopathy, Multiple Myeloma, Angiocentric immunoproliferative lesion, Lymphoid granulomatosis, Angioimmunoblastic lymphadenopathy, T-gamma lymphoproliferative disease, Waldenström's macroglobulinaemia, Alpha heavy chain disease, Gamma heavy chain disease, Franklin's disease, Immunoproliferative small intestinal disease, Mediterranean disease, Malignant immunoproliferative disease, unspecified, or Immunoproliferative disease NOS.

In still another embodiment, the present method can be used to treat or prevent an immune deficiency disease or disorder in a subject. The present method can be used to treat or prevent any suitable an immune deficiency disease or disorder in a subject. For example, the immune deficiency disease or disorder can be Agammaglobulinemia: X-Linked and Autosomal Recessive, Ataxia Telangiectasia, Chronic Granulomatous Disease and Other Phagocytic Cell Disorders, Common Variable Immune Deficiency, Complement Deficiencies, DiGeorge Syndrome, Hemophagocytic Lymphohistiocytosis (HLH), Hyper IgE Syndrome, Hyper IgM Syndromes, IgG Subclass Deficiency, Innate Immune Defects, NEMO Deficiency Syndrome, Selective IgA Deficiency, Selective IgM Deficiency, Severe Combined Immune, Deficiency and Combined Immune Deficiency, Specific Antibody Deficiency, Transient Hypogammaglobulinemia of Infancy, WHIM Syndrome (Warts, Hypogammaglobulinemia, Infections, and Myelokathexis), Wiskott-Aldrich Syndrome, Other Antibody Deficiency Disorders, Other Primary Cellular Immunodeficiencies, Severe combined immune deficiency (SCID), Common variable immune deficiency (CVID), Human immunodeficiency virus/acquired immune deficiency syndrome (HIV/AIDS), Drug-induced immune deficiency, Graft versus host syndrome, Primary Immune Deficiency Diseases (PIDDs), or Lymphopenia.

In still another embodiment, the present method can be used to treat or prevent an autoimmune disease or disorder. For example, the present method can be used to treat or prevent inflammation, autoimmune disease, paraneoplastic autoimmune diseases, cartilage inflammation, fibrotic disease and/or bone degradation, arthritis, rheumatoid arthritis, juvenile arthritis, juvenile rheumatoid arthritis, pauciarticular juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reter's Syndrome, SEA Syndrome (Seronegativity, Enthesopathy, Arthropathy Syndrome), juvenile dermatomyositis, juvenile psoriatic arthritis, juvenile scleroderma, juvenile systemic lupus erythematosus, juvenile vasculitis, pauciarticular rheumatoid arthritis, polyarticular rheumatoid arthritis, systemic onset rheumatoid arthritis, ankylosing spondylitis, enteropathic arthritis, reactive arthritis, Reter's Syndrome, SEA Syndrome (Seronegativity, Enthesopathy, Arthropathy Syndrome), dermatomyositis, psoriatic arthritis, scleroderma, systemic lupus erythematosus, vasculitis, myolitis, polymyolitis, dermatomyolitis, osteoarthritis, polyarteritis nodossa, Wegener's granulomatosis, arteritis, ploymyalgia rheumatica, sarcoidosis, scleroderma, sclerosis, primary biliary sclerosis, sclerosing cholangitis, Sjogren's syndrome, psoriasis, plaque psoriasis, guttate psoriasis, inverse psoriasis, pustular psoriasis, erythrodermic psoriasis, dermatitis, atopic dermatitis, atherosclerosis, lupus, Still's disease, Systemic Lupus Erythematosus (SLE), myasthenia gravis, inflammatory bowel disease (IBD), Crohn's disease, ulcerative colitis, celiac disease, multiple schlerosis (MS), asthma, COPD, Guillain-Barre disease, Type I diabetes mellitus, thyroiditis (e.g., Graves' disease), Addison's disease, Raynaud's phenomenon, autoimmune hepatitis, GVHD, transplantation rejection, and/or the like. However, autoimmune disease or disorder is a very active area of research, and further diseases or disorder may be identified as the present invention can be obtained by the treatment. In some embodiments, an autoimmune disease or disorder refers to a disease or disorder in which the immune system attacks its own proteins, cells, tissues and organs, etc. For example, in some human autoimmune diseases or disorders, human immune system attacks its own proteins, cells, tissues and organs, etc, including diseased proteins, cells, tissues and organs. A review of some autoimmune diseases or disorders and their list can be found in The Autoimmune Diseases (Rose and Mackay, 6th Edition, 2019, Academic Press). The present method can further comprise administering an effective amount of a second therapeutic agent for treating or preventing a proliferation disorder in a subject. For example, the present method can be used for treating or preventing a proliferation disease or disorder, e.g., a tumor or a cancer, in a subject and further comprise administering an anti-neoplasm substance to the subject.

To practice the method of the present invention, a modified IL-2 polypeptide, a polynucleotide, e.g., DNA, RNA or viral vector, a modified IL-2 polypeptide conjugate or a pharmaceutical composition, as described above, may be administered via any suitable route. For example, a modified IL-2 polypeptide, a polynucleotide, e.g., DNA, RNA or viral vector, a modified IL-2 polypeptide conjugate or a pharmaceutical composition, as described above, may be administered orally, parenterally, by inhalation, topically, rectally, nasally, buccally, vaginally, via an implanted reservoir, or other drug administration methods. The term “parenteral” as used herein includes subcutaneous, intracutaneous, intravenous, intramuscular, intraarticular, intraarterial, intrasynovial, intrasternal, intrathecal, intralesional and intracranial injection or infusion techniques.

A sterile injectable composition, such as a sterile injectable aqueous or oleaginous suspension, may be formulated according to techniques known in the art using suitable dispersing or wetting agents and suspending agents. The sterile injectable preparation may also be a sterile injectable solution or suspension in a non-toxic parenterally acceptable diluent or solvent. Among the acceptable vehicles and solvents that may be employed include mannitol, water, Ringer's solution and isotonic sodium chloride solution. Suitable carriers and other pharmaceutical composition components are typically sterile.

In addition, sterile, fixed oils are conventionally employed as a solvent or suspending medium (e.g., synthetic mono- or diglycerides). Fatty acids, such as oleic acid and its glyceride derivatives, are useful in the preparation of injectables, as are pharmaceutically acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Various emulsifying agents or bioavailability enhancers which are commonly used in the manufacture of pharmaceutically acceptable solid, liquid, or other dosage forms can also be used for the purpose of formulation.

A composition for oral administration may be any orally acceptable dosage form including, but not limited to, tablets, capsules, emulsions and aqueous suspensions, dispersions and solutions. In the case of tablets for oral use, commonly used carriers include lactose and corn starch. Lubricating agents, such as magnesium stearate, can also be added. For oral administration in a capsule form, useful diluents include lactose and dried corn starch. When aqueous suspensions or emulsions are administered orally, the active ingredient can be suspended or dissolved in an oily phase combined with emulsifying or suspending agents. If needed, certain sweetening, flavoring, or coloring agents can be added. A nasal aerosol or inhalation compositions can be prepared according to techniques well-known in the art of pharmaceutical formulation and can be prepared as solutions in, for example saline, employing suitable preservatives (for example, benzyl alcohol), absorption promoters to enhance bioavailability, and/or other solubilizing or dispersing agents known in the art.

In yet another aspect, the present invention is directed to an use of an effective amount of a modified IL-2 polypeptide, a polynucleotide, e.g., DNA, RNA or viral vector, or a modified IL-2 polypeptide conjugate, as described above, for the manufacture of a medicament for treating or preventing a disease or a disorder, e.g., a proliferation disease or disorder, in a subject.

### G. Methods for Expanding Various Immune Cells

In yet another aspect, the present invention is directed to a method of expanding a CD4⁺ helper cell, CD8⁺ effector naive and memory cell, Natural Killer (NK) cell, or Natural killer T (NKT) cell population, which comprises contacting a cell population with an effective amount of a modified IL-2 polypeptide, a polynucleotide, e.g., DNA, RNA or viral vector, a modified IL-2 polypeptide conjugate or a pharmaceutical composition, as described above, for a time sufficient to induce formation of a complex with an IL-2Rβγ, thereby stimulating the expansion of the T cell, NK cell, and/or NKT cell population.

In yet another aspect, the present invention is directed to a method of expanding a CD4⁺ helper cell, CD8⁺ effector naive and memory cell, Treg cells, Natural Killer (NK) cell, or Natural killer T (NKT) cell population, which comprises contacting a cell population with an effective amount of a modified IL-2 polypeptide, a polynucleotide, e.g., DNA, RNA or viral vector, a modified IL-2 polypeptide conjugate or a pharmaceutical composition, as described above, for a time sufficient to induce formation of a complex with an IL-2Rβγ, thereby stimulating the expansion of the T cell, Treg cell, NK cell, and/or NKT cell population with reduced cell death by 10% to 100%, e.g., with reduced cell death by 10%, 20%, 30%, 40%, 50%, 60%, 70%, 80%, 90%, 100% or any subrange thereof.

In one embodiment, the modified IL-2 polypeptide, a polynucleotide, e.g., DNA, RNA or viral vector, the modified IL-2 polypeptide conjugate or pharmaceutical composition, as described above, expands CD4⁺ T regulatory (Treg) cells by less than 20%, 19%, 18%, 17%, 16%, 15%, 14%, 13%, 12%, 11%, 10%, 9%, 8%, 7%, 6%, 5%, 4%, 3%, 2%, 1% or less in the CD3⁺ cell population compared to an expansion of CD4⁺ Treg cells in the CD3⁺ cell population contacted with a comparable IL-2 polypeptide comprising an amino acid sequence set forth in SEQ ID NO:1 or SEQ ID NO:2 without the substitution. In another embodiment, the modified IL-2 polypeptide, a polynucleotide, e.g., DNA, RNA or viral vector, modified IL-2 polypeptide conjugate or pharmaceutical composition, as described above, does not expand CD4⁺ Treg cells in the cell population. In still another embodiment, the ratio of the Teff cells to Treg cells in the cell population after incubation with the modified IL-2 polypeptide, a polynucleotide, e.g., DNA, RNA or viral vector, modified IL-2 polypeptide conjugate or pharmaceutical composition, as described above, is about or at least 2:1, 3:1, 4:1, 5:1, 6:1, 7:1, 8:1, 9:1, 10:1, 20:1, 50:1, 100:1 or more.

The present methods can be conducted in any suitable manner. In one embodiment, the present method is conducted in vivo. In another embodiment, the present method is conducted in vitro. In still another embodiment, the present method is conducted ex vivo.

In yet another aspect, the present invention is directed to an use of an effective amount of a modified IL-2 polypeptide, a polynucleotide, e.g., DNA, RNA or viral vector, or a modified IL-2 polypeptide conjugate, as described above, for the manufacture of a medicament for expanding a CD4⁺ helper cell, CD8⁺ effector naive and memory cell, Treg cell, Natural Killer (NK) cell, or Natural killer T (NKT) cell population in a cell population. In one embodiment, the present use is configured for expanding a CD4⁺ helper cell, CD8⁺effector naive and memory cell, Treg cell, Natural Killer (NK) cell, or Natural killer T (NKT) cell population in a subject.

### H. Examples

### Example 1. Design of the PEG Modified IL2 Muteins

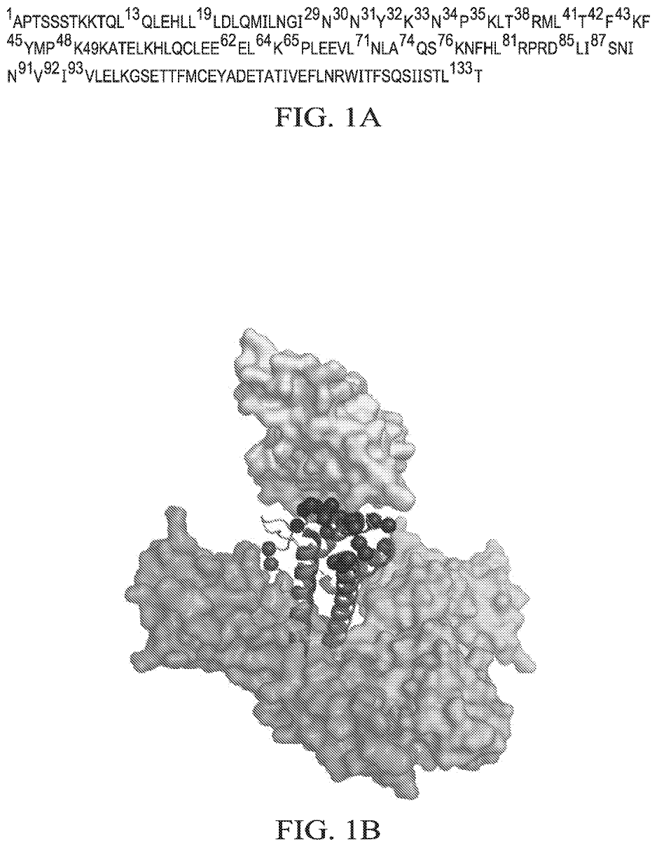

Selection of PEG attachment sites in IL-2. From human IL-2 peptide sequence, one of the amino acids from the list of “Site 1” (Table 1) are selected and substituted with cysteine, so that the mutein can be conjugated with maleimide-activated PEG reagents. The PEG conjugated muteins are expected to have extended half-life compared with native IL-2 molecule. These PEGylations sometimes also interfere with the binding to alpha unit of IL-2 receptor (IL-2Rα), while keep the binding to beta and gamma units (IL-2Rβ, IL-2Rγ) intact (FIG. 1, Table 1). All constructs are made on the background of wild-type human IL-2 with substitution C125S to remove this unpaired cysteine residue in IL-2 (called here rhIL-2).

TABLE 1
IL-2 mutations design

| Pegylated | IL-2Rα interaction | IL-2Rβ interaction |
|---|---|---|
| sites (Site 1) | sites (Site 2) | sites (Site 3) |
| N29C | R38 | Q13 |
| N30C | P65 | L19 |
| Y31C | F42 | R81 |
| K32C | E62 | L85 |
| N33C | Y45 | V91 |
| P34C | | S87 |
| K35C | | I92 |
| R38C | | V93 |
| T41C | | |
| F42C | | |
| K43C | | |
| Y45C | | |
| K48C | | |
| K49C | | |
| E62C | | |
| K64C | | |
| P65C | | |
| N71C | | |
| Q74C | | |
| K76C | | |

The final design of the pegylated IL-2 mutein molecules are expected to have reduced affinity for IL-2Rα, robust binding to IL-2Rβγ, and extended half-life in human and other animals.

Selection of additional mutation sites that disrupt IL-2Rα interaction or enhance IL-2Rβ. On top of the PEGylation modification described in step 1, additional modifications sometimes are introduced. The modifications carry at least one mutation that substitute an amino acid from the list of “Site 2” (Table1) with any other amino acids. Mutations at these sites reduced binding to IL-2Rα, while keep the IL-2Rβ and IL-2Rγ binding substantially intact (Table 1). The modification may also carry at least one mutation that substitutes an amino acid from the list of “Site 3” (Table 1) with any other amino acids to enhance its binding to IL-2Rβ.

### Example 2. Production and purification of IL-2 muteins

cDNAs encoding IL-2 muteins were synthesized and cloned into pcDNA3.1 (−) vector. HEK293F cells were transient transfected with PEI MAX (Polysciences) and cultured for 96 hours. The supernatants were harvested by centrifugation of the culture at 4000×g for 20 minutes.

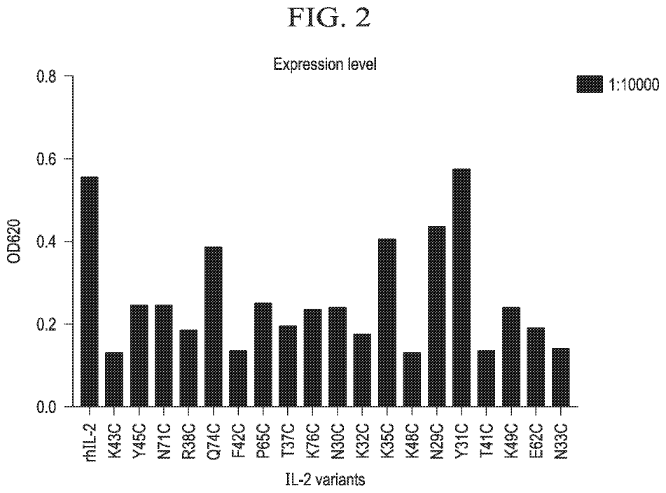

HEK-Blue™ IL-2 reporter cells (InvivoGen, hkb-il2) were used to determine IL-2 expression levels. Upon IL-2 stimulation, HEK-Blue™ IL-2 cells trigger the activation of STATS and the subsequent secretion of SEAP. The levels of STATS-induced SEAP can be readily monitored using QUANTI-Blue™. The cells were seeded at 100,000 cells/well in 100 μl, then 100 μl of rhTL-2 or IL-2 muteins were added to the wells. 20-24 hours later, 180 μl of supernatants were collected and mixed with 20 μl of Quantiblue in a flat bottom plate. After 90 min incubation at 37° C., absorbance was read at 620 nm. FIG. 2. Indicated that using 10,000 diluted culture supernatants, IL-2 variants had different detectable expression levels.

Standard protein purification techniques were used to isolate the proteins of interest from the supernatant. In brief, the protein of interest was captured by cOmplete® His-Tag Purification column (Roche) and polished by Superdex 75 Increase column (GE Healthcare). Purified proteins were eluted in buffer containing 0.1M IVIES and 150 mM NaCl, pH 6.0 and were stored in −80° C. for further use.

### Example 3. PEGylation of IL-2 Muteins

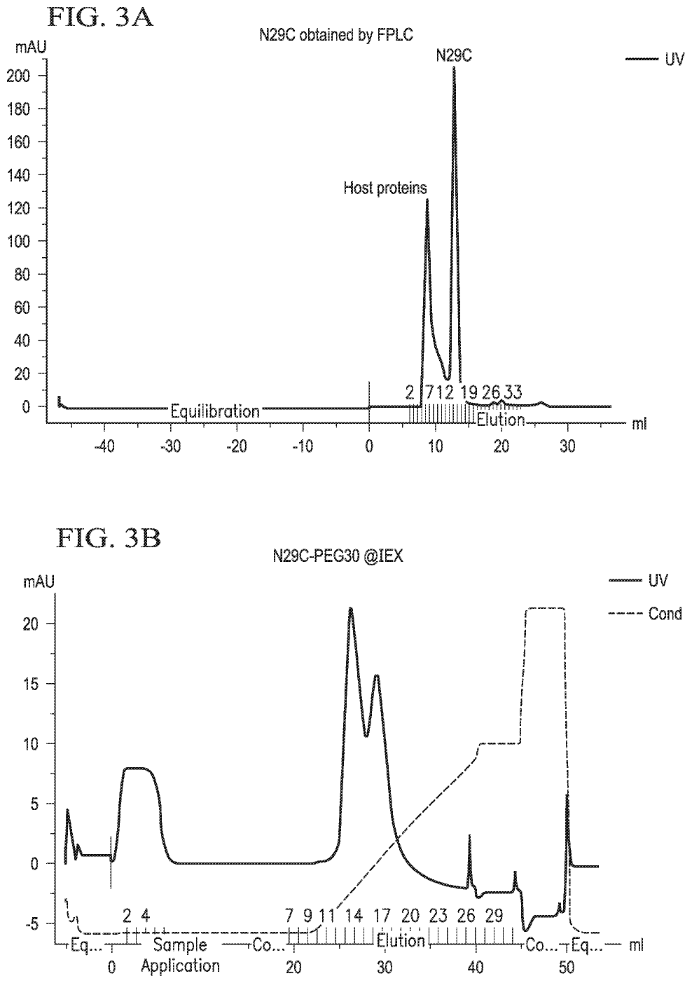

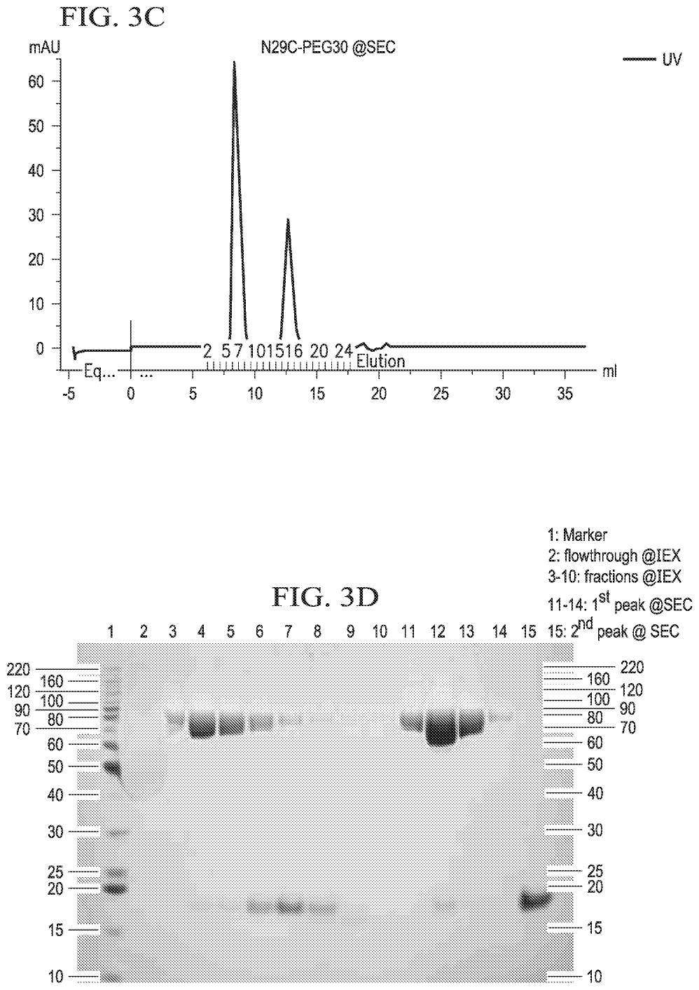

Purified IL-2 muteins (1 mg/ml) were reduced by 5 mM TCEP (Thermo Fisher) at room temperature for 15 min and then reacted with a 50-fold molar excess of maleimide-PEG 20K (Laysan Bio) for 30 minutes at room temperature. The reaction was stopped by adding L-cysteine (Sigma) to a 2-fold molar excess over maleimide-PEG 20k. PEG-conjugates were further purified by SP Sepharose FF column which was followed by Superdex 75 Increase column (GE Healthcare). Representative chromatogram and SDS-PAGE analysis of the purification process were shown in FIG. 3.

### Example 4. Binding of IL-2 Muteins and PEG-Conjugates with IL-2 Receptors

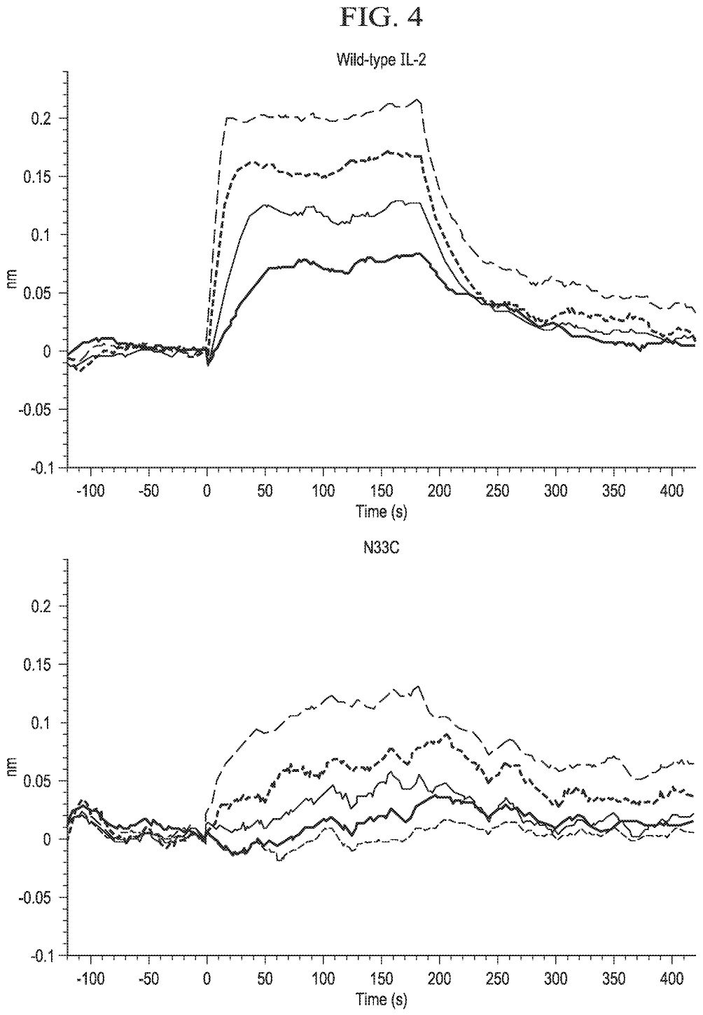

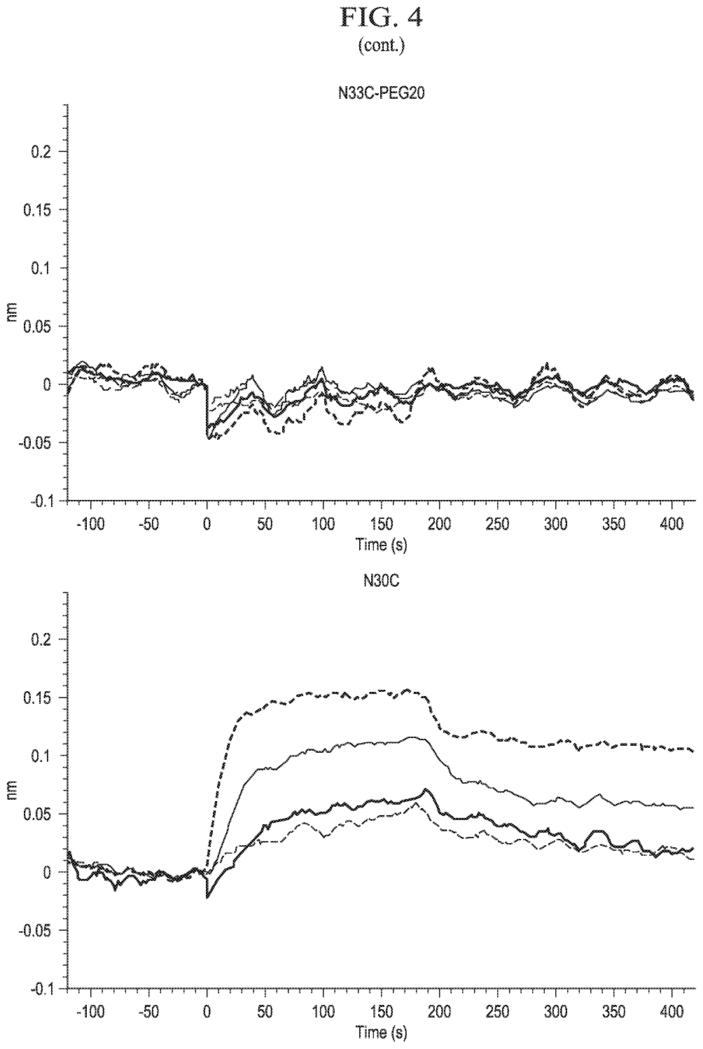

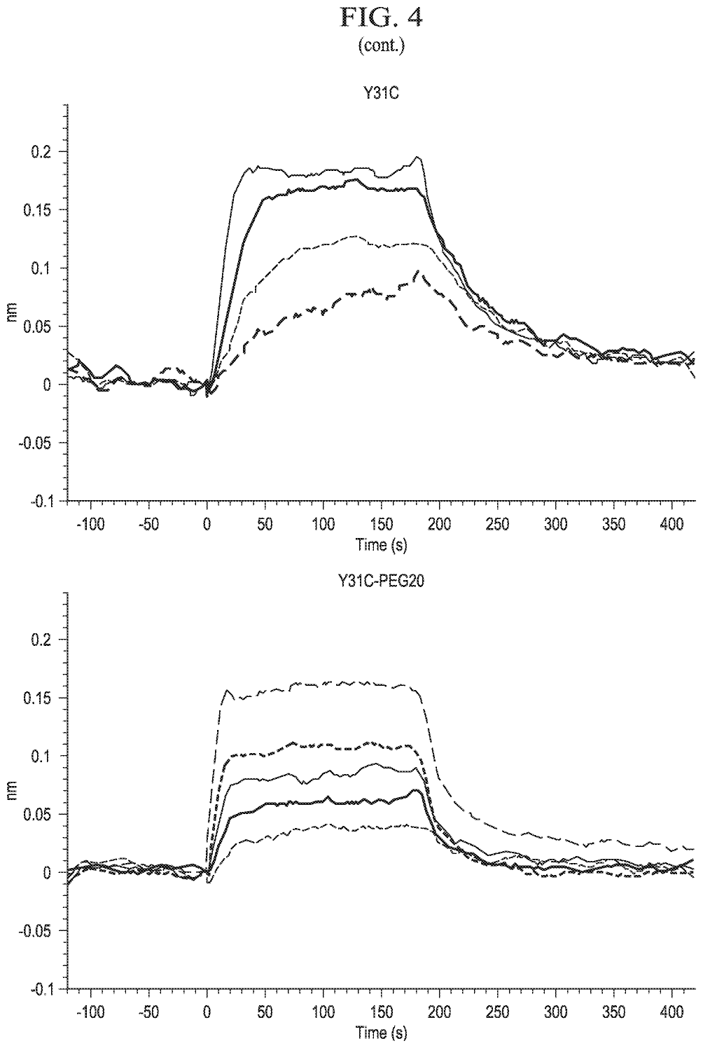

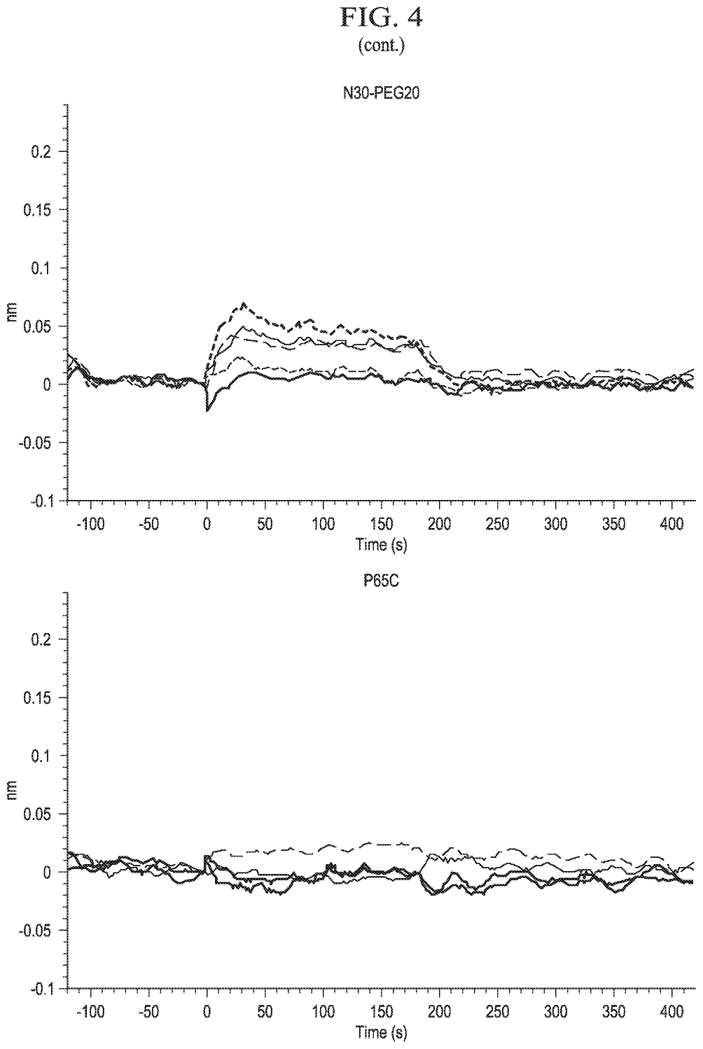

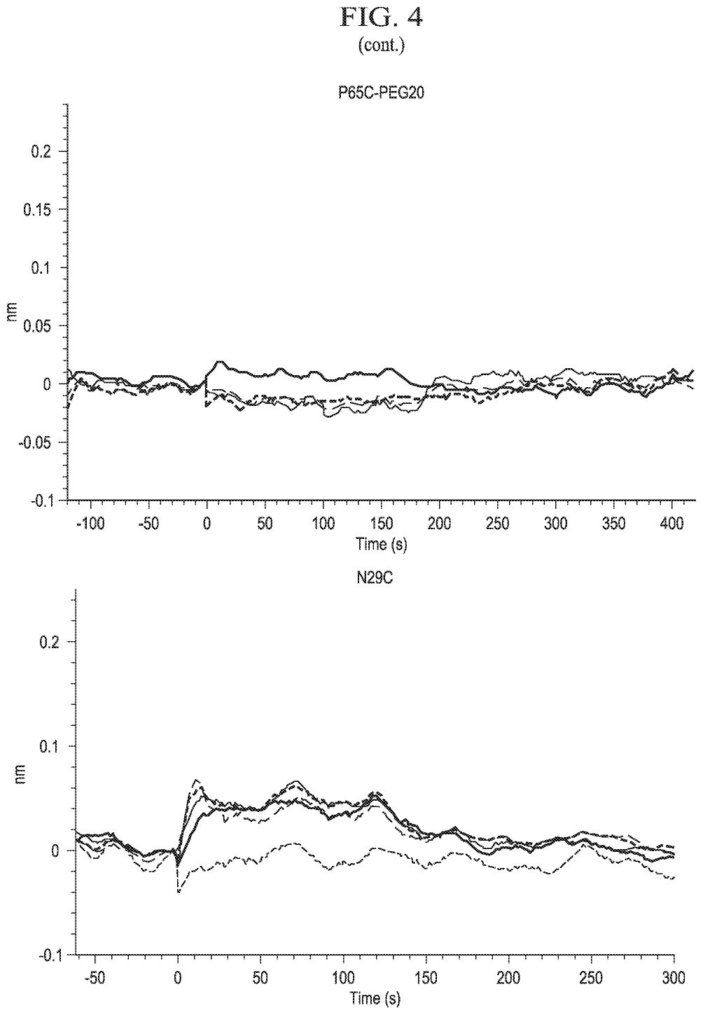

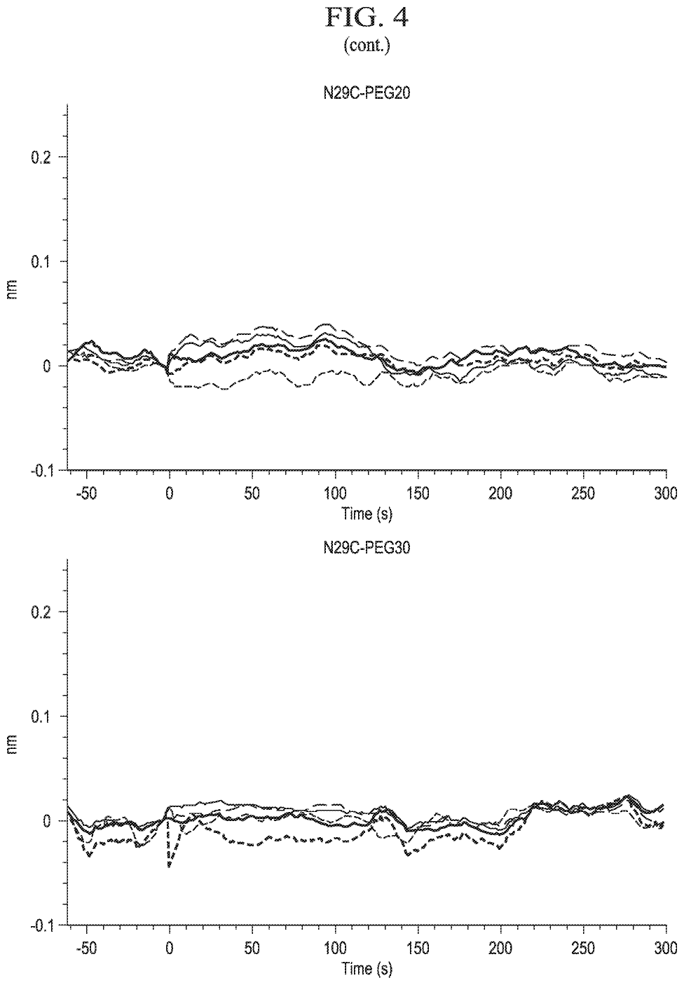

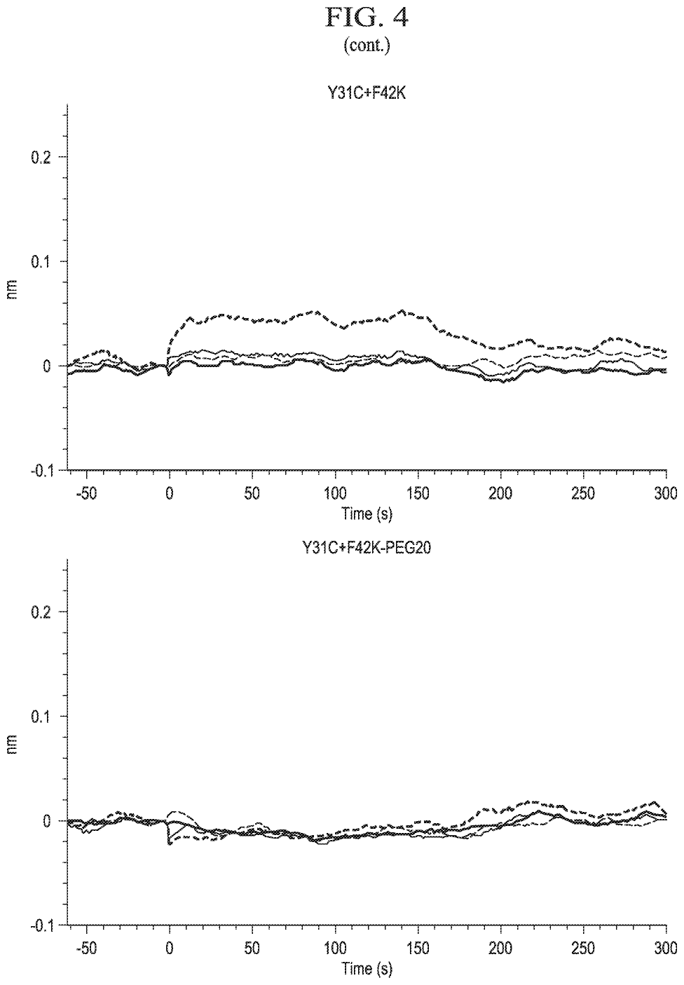

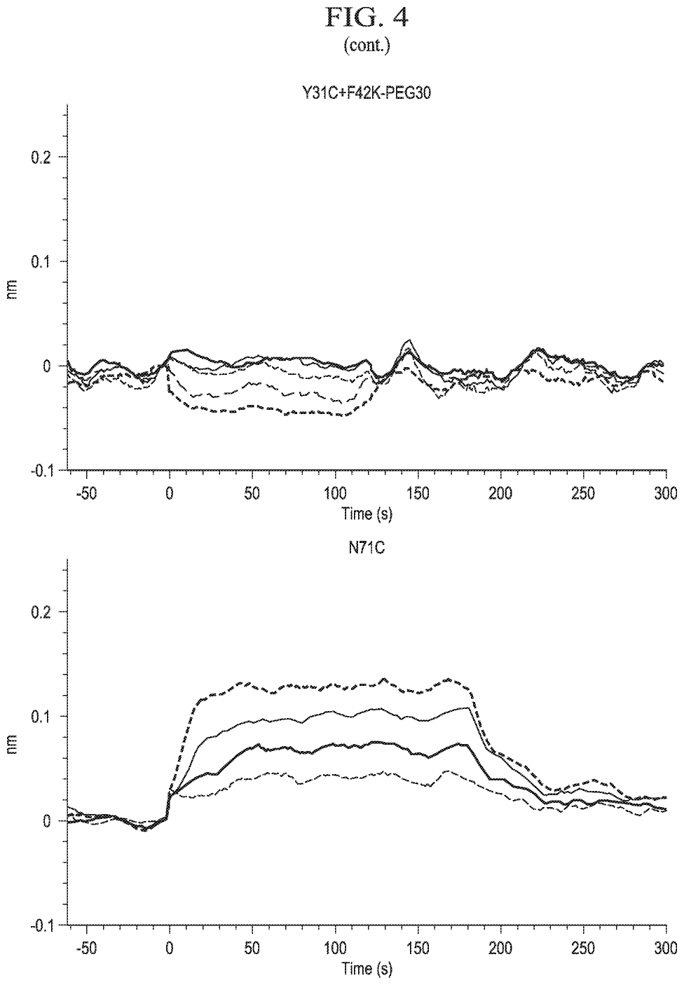

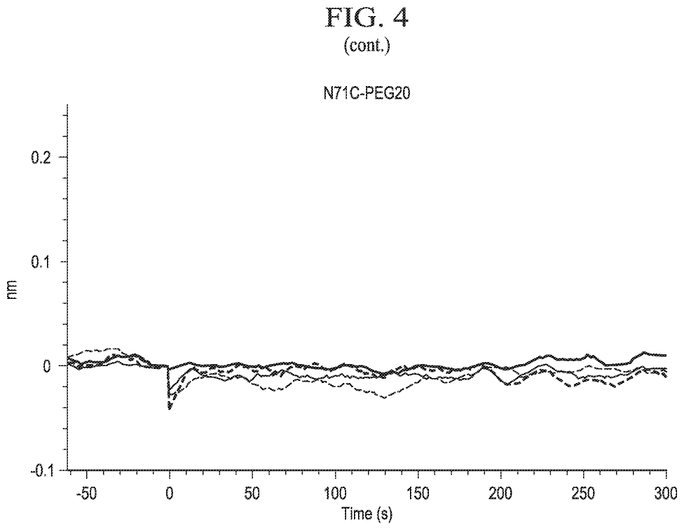

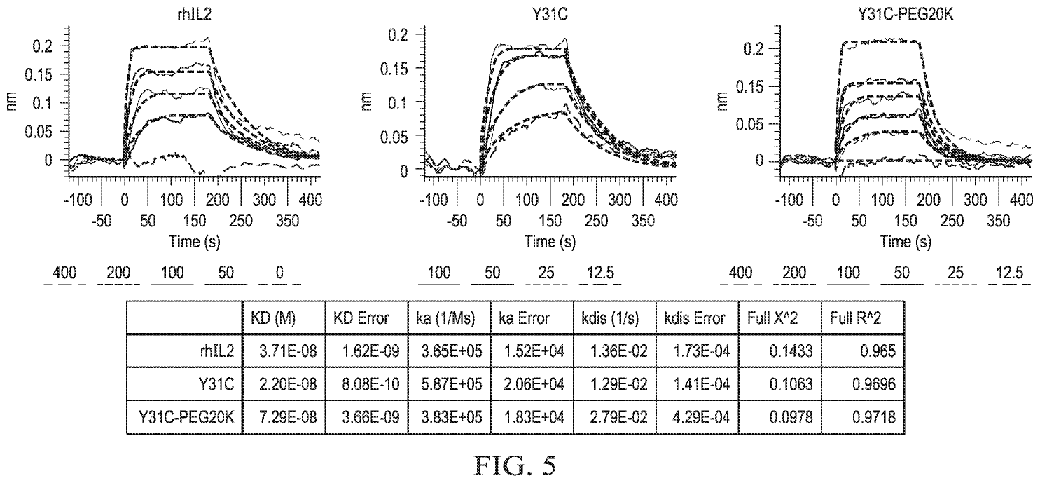

Binding of purified IL-2 muteins or PEG-conjugates with IL-2 receptors was determined by Octet QKe (ForteBio). IL-2Rα or IL-2Rβ in human Fc fusion protein format (ACROBiosystems) were captured on anti-Human IgG Fc Capture (AHC) sensors. After the baseline was established in 1× Kinetics buffer, the sensors were dipped into wells containing serial diluted rhIL-2, muteins or PEG-conjugates to measure association constants. Dissociation was detected following transfer of sensors into wells containing buffer alone. Data were collected and analyzed by Octet User Software. For analysis of the kinetics constants, 1:1 curve fitting model was used. Table 2 shows kinetic parameters for IL-2 variants binding with individual IL-2 receptor subunit. Typical sensorgrams of the binding were showed in FIG. 4. Most PEGylated muteins showed reduced or abolished binding with IL-2Rα.

TABLE 2
Kinetic constants of IL-2 variants interaction with IL-2Rα

| | Kₒₙ (M⁻¹S⁻¹) | K_{off} (S⁻¹) | K_{D} (μM) |
|---|---|---|---|
| rhIL-2 | 6.80 ± 0.26 × 10⁵ | 2.03 ± 0.02 × 10⁻² | 0.030 ± 0.001 |
| Y31C-PEG20 | 3.83 ± 0.18 × 10⁵ | 2.79 ± 0.04 × 10⁻² | 0.073 ± 0.004 |
| K35C-PEG20 | ND | ND | ND |
| R38C-PEG20 | ND | ND | ND |
| P65C-PEG20 | ND | ND | ND |
| T41C-PEG20 | ND | ND | ND |
| N30C-PEG20 | 5.93 ± 0.89 × 10⁵ | 6.97 ± 0.35 × 10⁻² | 0.117 ± 0.018 |
| N33C-PEG20 | ND | ND | ND |
| Y31C- | ND | ND | ND |
| PEG20 + F42K | | | |

Note:
ND = not detectable.

### Example 5. Surface Binding of IL2 Muteins on IL2Rαβγ Expressing Cells

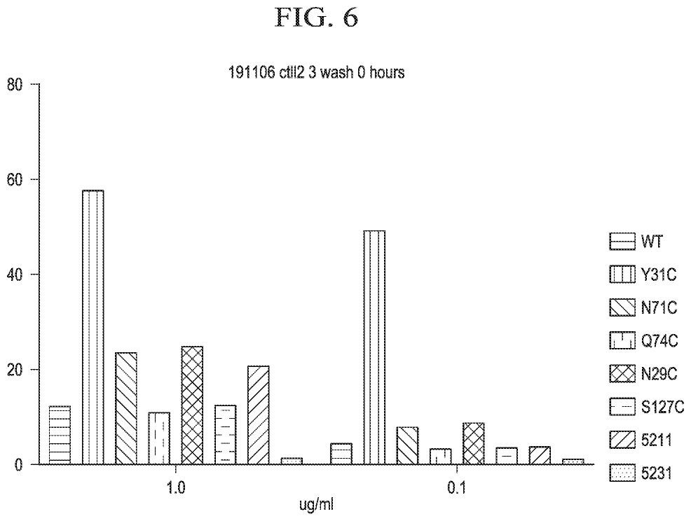

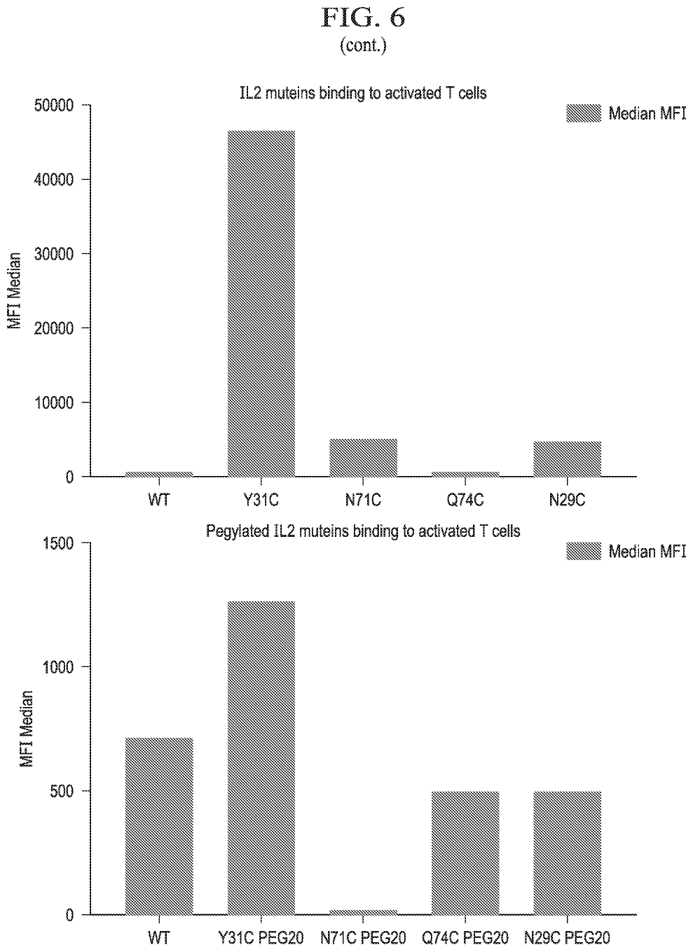

Two different IL2Rαβγ expressing cells were tested for this analysis: 1. CTLL2 cells; 2. IL2Rα+ T cells generated by anti-CD3/CD28 Dynabeads reactivated human T cells from PBMC (at least 90% of cells were positive for IL2Rα). Either CTLL2 or IL2Rα+ T cells were collected and resuspended in cold binding buffer (FBB, 5% FBS in DPBS) at 2-4 million cells/ml. Histagged IL-2 and mutants were added to the cell suspension, mixed and kept at 4° C. for 40 min. The cells were washed once in wash buffer (FWB, 1% FBS in DPBS). Resuspend cell pellets in FBB were reacted with 1:100 anti-His-APC (BioLegend 362605), at room temperature for 15 min. Cells were washed with 120 ul FWB then resuspend for flow cytometry analysis. Y31C and Y31C-PEG20 both showed enhanced binding on CTLL2 cells and IL2Rα positive human T cells. (See FIG. 6.)

### Example 6. T Cell Activity of PEGylated IL-2 Muteins

Frozen PBMCs were defrost in AIM-V media (ThermoFisher) without serum and kept at 37° C. for 2-4 h before the experiment. 5×10⁵ cells/well were seeded in 96 well plate. The different IL-2 muteins were added on top of the cells at 4C, to avoid phosphorylation of STAT-5 in different time points. The cells were mixed with the muteins and incubated at 37 C for 15 min. The rest of the protocol was performed at room temperature. After centrifugation, the cell pellet was stained for extracellular markers (1:300—anti-human CD4 FITC, CD8 APC, CD25 BV650, R45RA BV421, BioLegend) and Fixable Viability Dye (1:1000— eFluor 780, ThermoFisher) for 15 min in 50 μL of Staining Buffer (PBS+1% FBS+2 mM EDTA). The cells were washed with 200 μL of wash buffer (PBS+1% FBS) and spun. The cells pellet was fixed with 200 μL of 1× Fixation Buffer (FoxP3/Transcription Factor Staining Buffer Set, eBioscience) for 30 min, in the dark. The cells were spun and permeabilized with 100 μL of cold 100% methanol at 4C, overnight. After this period, 100 μL of wash buffer were added, the cells were spun and stained with 50 μL anti-human P STATS-PE (1:80, BioLegend) for 30 min at room temperature, in the dark. 250 μL of wash buffer were added, the cells were spun and resuspend in 110 μL of Staining Buffer. The indicated surface markers and the phosphorylation of STAT-5 from CD8⁺CD45RA⁺CD25^{low} naïve (IL-2Rβγ expressing T cells) and CD4⁺CD45RA⁻CD25^{high} (IL-2Rαβγ expressing T cells) T cells were assessed by flow cytometry (NovoCyte, ACEA Biosciences).

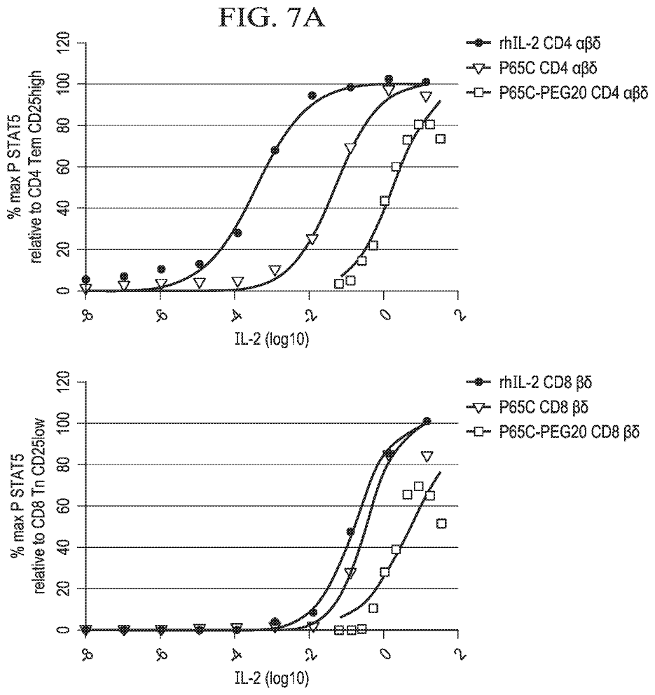

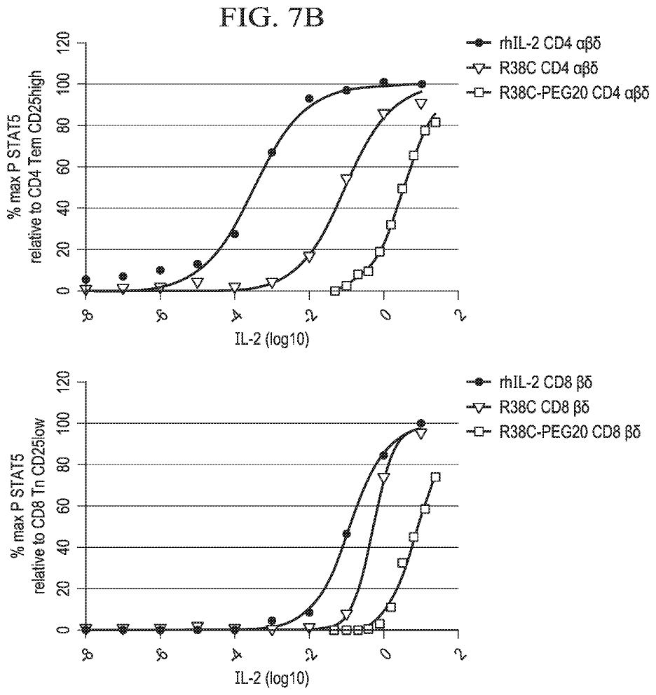

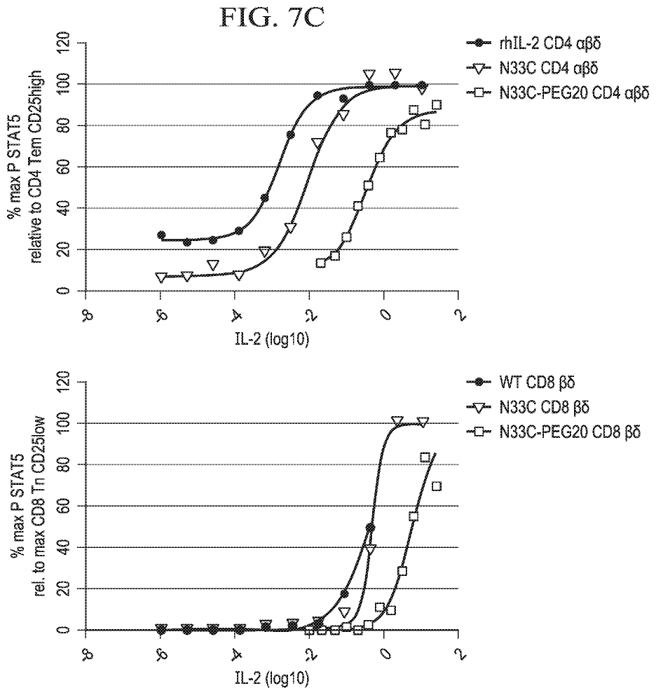

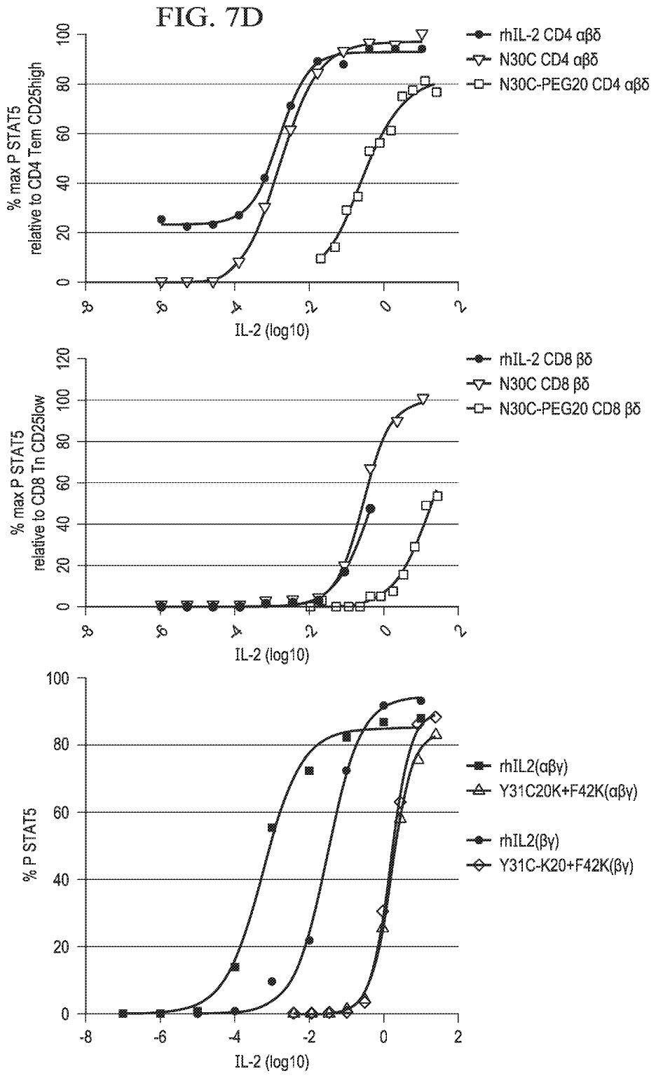

FIG. 7 shows the dose-response phosphorylation of STATS in different IL-2 muteins and respective pegylated proteins. When compared to the rhIL-2 protein, Pegylated IL-2 muteins showed drastically reduced activity on cells expressing IL-2Rαβγ while their activities to IL-2Rβγ expressing T cells are largely intact (Table 3). The favorable bioselectivity of the Pegylated muteins towards T cells expressing IL-2Rβγ over IL-2Rαβγ was also demonstrated by the ratio of EC50 on the two different cell populations.

TABLE 3
T cell activities of pegylated IL2 muteins

| EC50 | CD25-CD8+ naïve T | CD25+CD4+ T | |
|---|---|---|---|
| ug/ml | (IL-2Rβγ) | (IL-2Rαβγ) | Ratio |
| rhIL-2 | 0.4379 | 0.0016 | 273.7 |
| N33-P20 | 6.342 | 0.31 | 20.5 |
| N30-P20 | 17.3 | 0.26 | 66.5 |
| P65-P20 | 3.976 | 1.361 | 2.92 |
| R38-P20 | 8.186 | 3.389 | 2.42 |
| Y31C-PEG20 + | 1.6 | 1.65 | 1.03125 |
| F42K | | | |

### Example 7. PK Study in C57BL/6 Mice

Pharmacokinetics studies of P65C-PEG20 or Y31C-PEG20+F42K were conducted in C57BL/6 mice. The following used P65C-PEG20 as example. 3 mice were used for each time point blood collection. Each mouse was administered with a single IV dose of 0.56 mg/kg P65C-PEG20. Blood samples were collected at 0.033, 0.083, 0.17, 0.5, 1, 4, 24, 48, 72, and 96 h post-dose. Blood were allowed to clot at room temperature prior to be processed by centrifugation at 5000 rpm for 10 min. Sera were collected, frozen in dry ice and kept at −80° C. until ELISA analysis.

ELISA were conducted in two phases. For the 1^{st} phase, most samples were diluted 10 times, except for early time point samples from 2 min to 30 min post dose. They were diluted 100-1,000 times. Diluted samples were added to ELISA plates coated with rabbit anti-IL-2 antibody P600 (ThermoFisher), and detected with biotin conjugated monoclonal IL-2 antibody M600B (ThermoFisher). For samples collected from 1 hr and later, samples were further tested with High Sensitivity IL-2 Human ELISA Kit (ThermoFisher) to detect low level of IL-2. All tests were done in duplicates. ELISA reading were converted to concentrations using standard curves of corresponding IL-2 constructs and Pade(1,1) approximant model (Prism).

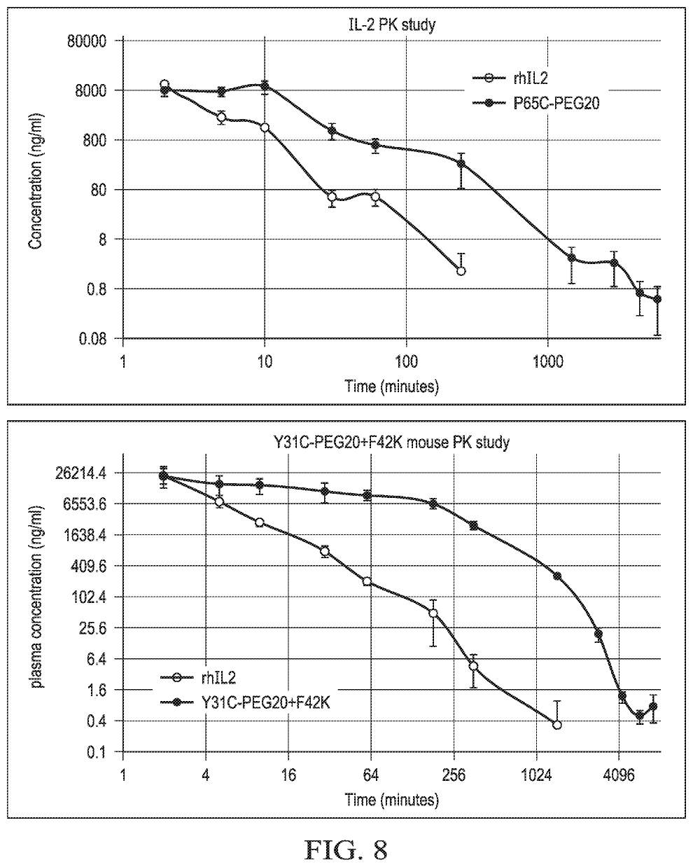

The PK parameter calculations were analyzed by non-compartmental method using Phenix WinNonLin Version 8.1 software (Certara USA, Inc., Princeton, N.J., USA) (FIG. 8). The serum concentration-time profile of P65C-PEG20 in mice was similar to what was reported for Aldesleukin [REF-1]. The terminal half-life (t_{1/2}) and mean residence time (MRT_{inf}) of P65C-PEG20 were 23.2 h and 2.85 h, respectively (Table 4), whereas those of rhIL-2 were 4.0 h and 0.20 h, respectively [REF-1]. The area under the concentration-time curve (AUCₗₐₛₜ) of P65C-PEG20 was 8051 h*ng/mL at the 0.56 mg/kg dose, whereas the AUC of Aldesleukin was 1380 h*ng/mL at a 0.8 mg/kg dose [REF-1]. P65C-PEG20 exhibited longer terminal half-life (5.8-fold) and residence time (14.2-fold) and higher exposure (8.3-fold, dose normalized) than Aldesleukin. REF-1: Charych D, Khalili S, Dixit V, Kirk P, Chang T, Langowski J, et al. (2017) Modeling the receptor pharmacology, pharmacokinetics, and pharmacodynamics of NKTR-214, a kinetically-controlled interleukin-2 (IL2) receptor agonist for cancer immunotherapy. PLoS ONE 12(7): e0179431.

TABLE 4
PK parameters of IL2 P65C-PEG20

| | AUClast | HL_Lambda_z | Tmax | Cmax | C0_1^{a} | C0_2^{b} | Vz_pred | Cl_pred | AUCINF_pred | MRTINF_pred |
|---|---|---|---|---|---|---|---|---|---|---|
| Group | (hr*ng/ml) | (hr) | (hr) | (ng/ml) | (ng/ml) | (ng/ml) | (ml/kg) | (ml/hr/kg) | (hr*ng/ml) | (hr) |
| P65C- | 7919.6 | 3.09 | 0.17 | 9863.7 | 8982.9 | 27538.5 | 4489.2 | 1008.3 | 7934.8 | 1.95 |
| PEG20 | | | | | | | | | | |
| rhIL2 | 1530. | 0.67 | 0.03 | 10622.4 | 29416.2 | 29416.2 | 5073.3 | 5219.6 | 1532.7 | 0.14 |

### Example 8. Ex Vivo Amplification of T Cells and NK Cells

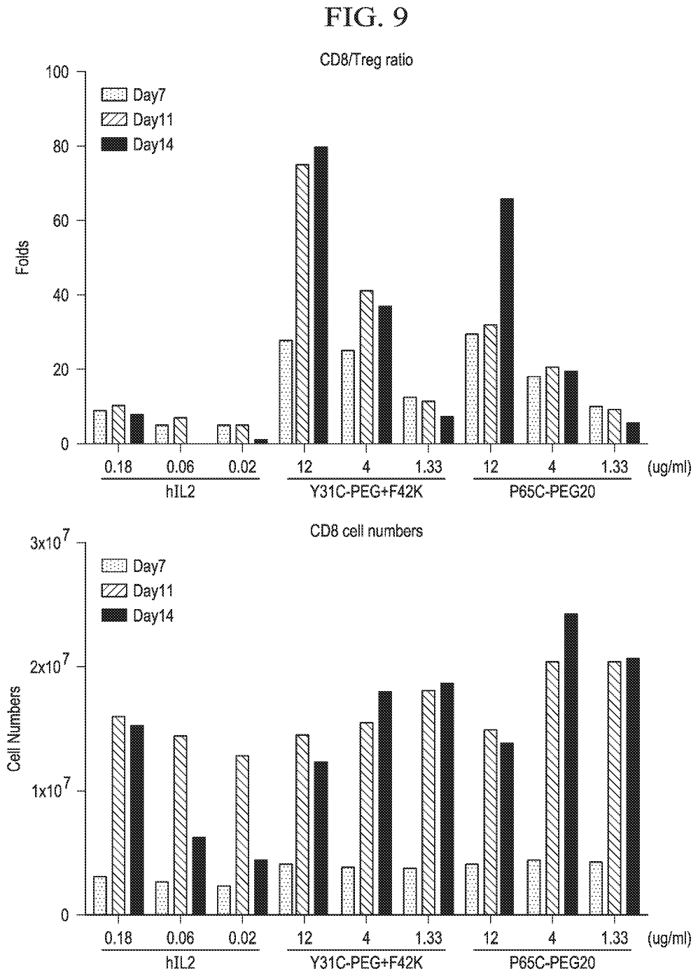

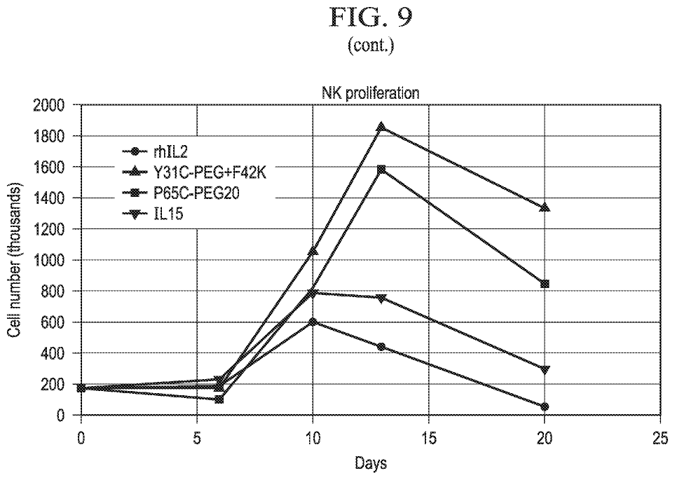

T cell proliferation. PBMC were thawn and grown in AIM V, 5% FBS, 5 ng/ml OKT3 and IL-2 or muteins at designated concentrations at 5 million cells/ml. Starting from day 5, cells were split every 3-4 days with media and IL-2 refresher. From Day 7, every 2-3 days, cells were stained and counted for total cells and subtypes of lymphocytes. Ex vivo amplified T cells have significantly reduced Treg and enhanced CD8 T/Treg ratio in the presence of P65C-PEG20 or Y31C-PEG20+F42K compared with rhIL2 (FIG. 9).

NK cell proliferation. PBMC were thawn and grown in AIM V, 5% FBS, IL-2 or muteins at designated concentrations at 5 million cells/ml in 24 well plate, with 0.5 ml/well. Cells were split every 2-3 days with media and IL-2 refresher. From Day 7, every 3-6 days, cells were stained and counted for total cells and subtypes of lymphocytes. Compared with rhIL2 or rhIL5, P65C-PEG20 or Y31C-PEG20+F42K promoted better proliferation of NK cells (FIG. 9).

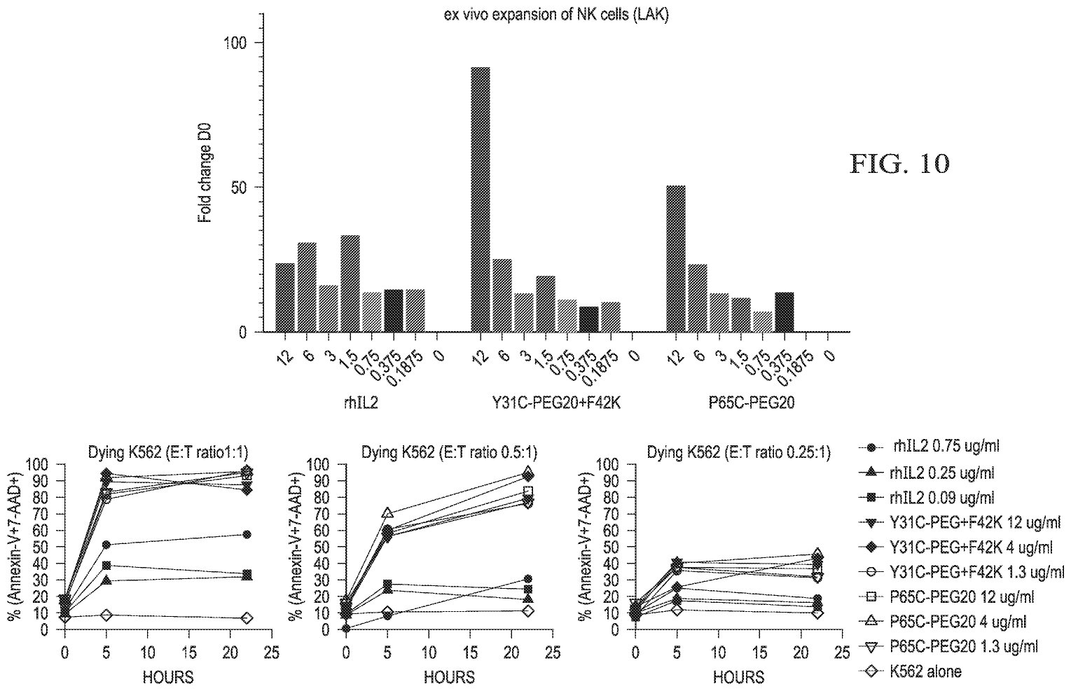

LAK cell cytotoxicity. PBMC derived LAK cells cultured for 2-4 weeks were used as effector cells. K562 stained with CFSE and grown overnight were used as target cells. Mix 30,000 K562 with different amount of LAK cells in the wells of 96 well U bottom plate. At different time points after co-culture, measure K562 cell viability by staining with Annexin V 7-AAD, and count CFSE+Annexin V+ population. P65C-PEG20 or Y31C-PEG20+F42K activated LAK cells showed enhanced proliferation and enhanced cytotoxicity to target K562 cells compared with rhIL2 (FIG. 10).

### I. References

The cited references are listed below.
1. Pachella L A, Madsen L T, Dains J E. The Toxicity and Benefit of Various Dosing Strategies for Interleukin-2 in Metastatic Melanoma and Renal Cell Carcinoma. J Adv Pract Oncol. 2015; 6(3):212-221.
2. Lotze, M. T., Frana, L. W., Sharrow, S. O., Robb, R. J., & Rosenberg, S. A. (1985). In vivo administration of purified human interleukin 2. I. Half-life and immunologic effects of the Jurkat cell line-derived interleukin 2. The Journal of Immunology, 134(1), 157 LP— 166.
3. Boyman, O., Krieg, C., Létourneau, S., & Pantaleo, G. (2009). Insight into Mechanism of IL-2-Induced Toxicity Provides Rationale for Improved Treatment Strategy using IL-2/mAb Complexes (38.8). The Journal of Immunology, 182(1 Supplement), 38.8 LP-38.8.
4. Maiser, B., Dismer, F. and Hubbuch, J. (2014), Optimization of random PEGylation reactions by means of high throughput screening. Biotechnol. Bioeng., 111: 104-114. doi:10.1002/bit.25000
5. Aldesleukin. DrugBank. (DB00041 (BTD00082, BIOD00082)):DB00041 (BTD82, BIOD82). DB00041 (BTD00082, BIOD00082).

1. A modified interleukin 2 (IL-2) polypeptide, which comprises an amino acid sequence set forth in SEQ ID NO:1 or SEQ ID NO:2 and a substitution with a natural amino acid or an unnatural amino acid at a position selected from the group consisting of Q13, L19, N29, N30, Y31, K32, N33, P34, K35, T37, R38, T41, F42, K43, Y45, K48, K49, E62, K64, P65, N71, Q74, K76, R81, L85, S87, V91, I92, V93 and a combination thereof, wherein said modified IL-2 polypeptide:
a) is configured to be unconjugated or conjugated to a water-soluble polymer, a lipid, or a polypeptide, e.g., a protein or a peptide; and/or
b) has reduced binding to an interleukin 2 receptor α (IL-2Rα) compared to a comparable IL-2 polypeptide comprising an amino acid sequence set forth in SEQ ID NO:1 or SEQ ID NO:2 without the substitution; and/or
c) has reduced receptor signaling potency to IL-2Rαβγ compared to a comparable IL-2 polypeptide comprising an amino acid sequence set forth in SEQ ID NO:1 or SEQ ID NO:2 without the substitution, and/or
d) has increased ratio of signaling potency to IL-2Rβγ over signaling potency to IL-2Rαβγ (i.e., increased ratio of signaling potency to IL-2Rβγ/signaling potency to IL-2Rαβγ) compared to a comparable IL-2 polypeptide comprising an amino acid sequence set forth in SEQ ID NO:1 or SEQ ID NO:2 without the substitution, and/or
e) has enhanced receptor signaling potency to IL-2Rβγ compared to a comparable IL-2 polypeptide comprising an amino acid sequence set forth in SEQ ID NO:1 or SEQ ID NO:2 without the substitution, and/or
provided that when said modified IL-2 polypeptide comprises a substitution with an unnatural amino acid, said modified IL-2 polypeptide comprises a substitution at a position selected from the group consisting of N29, N30, Y31, K32, N33, P34, K35, R38, T41, F42, K43, Y45, K48, K49, E62, K64, P65, N71, Q74, K76 and a combination thereof, and a substitution with a natural amino acid or an unnatural amino acid at a position within IL-2Rα interaction region, IL-2Rβ interaction region and/or IL-2Rγ interaction region, and
provided that said modified IL-2 polypeptide has at least about 80% sequence identity in the region of amino acid residues 10-25, 80-100 and/or 100-134 to the corresponding region of a comparable IL-2 polypeptide comprising an amino acid sequence set forth in SEQ ID NO:1 or SEQ ID NO:2 without the substitution, and said modified IL-2 polypeptide has at least about 50% sequence identity to a comparable IL-2 polypeptide comprising an amino acid sequence set forth in SEQ ID NO:1 or SEQ ID NO:2 without the substitution.

2. The modified IL-2 polypeptide of claim 1, which comprises a substitution with lysine, cysteine, histidine, arginine, aspartic acid, glutamic acid, serine, threonine, alanine, tryptophan, isoleucine, phenylalanine, or tyrosine at a position selected from the group consisting of Q13, L19, N29, N30, Y31, K32, N33, P34, K35, T37, R38, T41, F42, K43, Y45, K48, K49, E62, K64, P65, N71, Q74, K76, R81, L85, S87, V91, I92, V93 and a combination thereof.

3. The modified IL-2 polypeptide of claim 1 or 2, which:
a) comprises a substitution with a natural amino acid at a position selected from the group consisting of N29, N30, Y31, K32, N33, P34, K35, R38, T41, F42, K43, Y45, K48, K49, E62, K64, P65, N71, Q74, K76 and a combination thereof, and is configured to be conjugated to a water-soluble polymer, a lipid, a protein, or a peptide at the position selected from the group consisting of N29, N30, Y31, K32, N33, P34, K35, R38, T41, F42, K43, Y45, K48, K49, E62, K64, P65, N71, Q74, K76 and a combination thereof; and/or
b) comprises a substitution with a natural amino acid at a position selected from the group consisting of N29, N30, Y31, K32, N33, P34, K35, R38, T41, F42, K43, Y45, K48, K49, E62, K64, P65, N71, Q74, K76 and a combination thereof, and is configured to be conjugated to a water-soluble polymer, a lipid, a protein, or a peptide at the N terminal and/or C terminal of the polypeptide.

4. The modified IL-2 polypeptide of claim 3, which:
a) comprises a substitution with lysine, cysteine, histidine, arginine, aspartic acid, glutamic acid, serine, threonine, alanine, tryptophan, isoleucine, phenylalanine, or tyrosine at a position selected from the group consisting of N29, N30, Y31, K32, N33, P34, K35, R38, T41, F42, K43, Y45, K48, K49, E62, K64, P65, N71, Q74, K76 and a combination thereof; and/or
b) comprises a substitution with lysine, cysteine, histidine, arginine, aspartic acid, glutamic acid, serine, threonine, alanine, tryptophan, isoleucine, phenylalanine, or tyrosine at a position selected from the group consisting of N30, Y31, N33, P34, K35, R38, T41, K43, K48, K49, K64, P65, N71, Q74, K76 and a combination thereof.

5. The modified IL-2 polypeptide of claim 4, which:
a) comprises a substitution with cysteine at a position selected from the group consisting of N29, N30, Y31, N33, P34, K35, R38, T41, K43, K48, K49, K64, P65, N71, Q74, K76 and a combination thereof;
b) comprises a substitution with cysteine at a position selected from the group consisting of N29, Y31, K35, P65, N71, Q74 and a combination thereof;
c) comprises a substitution with any amino acid at a position of Y31, N29 or a combination thereof;
d) comprises a substitution with cysteine, serine or alanine at a position of Y31, N29 or a combination thereof;
e) comprises a substitution with cysteine at a position of Y31;
f) comprises a substitution with cysteine at a position of N29; and/or
g) comprises a substitution with cysteine at a position of P65.

6. The modified IL-2 polypeptide of any of claims 3-5, which further comprises a substitution with a natural amino acid or an unnatural amino acid at a position within IL-2Rα interaction region, IL-2Rβ interaction region and/or IL-2Rγ interaction region.

7. The modified IL-2 polypeptide of claim 6, which further comprises a substitution with a natural amino acid at a position within IL-2Rα interaction region.

8. The modified IL-2 polypeptide of claim 7, which comprises a substitution with a natural amino acid at a position selected from the group consisting of R38, F42, Y45, E62, P65 and a combination thereof.

9. The modified IL-2 polypeptide of claim 8, which comprises a substitution with lysine, cysteine, histidine, arginine, aspartic acid, glutamic acid, serine, threonine, alanine, tryptophan, isoleucine, phenylalanine, or tyrosine at a position selected from the group consisting of R38, F42, Y45, E62, P65 and a combination thereof.

10. The modified IL-2 polypeptide of claim 6 or 9, which:
a) comprises a substitution with cysteine at a position selected from the group consisting of R38, F42, Y45, E62, P65 and a combination thereof;
b) comprises a substitution with alanine, lysine or serine at a position of F42;
c) comprises a substitution with alanine at a position of F42;
d) comprises a substitution with serine at a position of F42;
e) comprises a substitution with lysine at a position of F42;
f) comprises a substitution with alanine, histidine or serine at a position of Y45;
g) comprises a substitution with alanine at a position of Y45;
h) comprises a substitution with histidine at a position of Y45;
i) comprises a substitution with alanine, aspartic acid or serine at a position of R38;
j) comprises a substitution with aspartic acid at a position of R38;
k) comprises a substitution with alanine at a position of P65;
l) comprises a substitution with serine at a position of P65;
m) comprises a substitution with alanine at a position of E62; and/or
n) comprises a substitution with lysine at a position of F42, a substitution with cysteine at position of Y31, or a combination thereof.

11. The modified IL-2 polypeptide of claim 6, which further comprises a substitution with a natural amino acid at a position within IL-2Rβ interaction region.

12. The modified IL-2 polypeptide of claim 11, which comprises a substitution with a natural amino acid at a position selected from the group consisting of Q13, L19, R81, L85, S87, V91, I92, V93 and a combination thereof.

13. The modified IL-2 polypeptide of claim 12, which comprises a substitution with lysine, cysteine, histidine, arginine, aspartic acid, glutamic acid, serine, threonine, alanine, tryptophan, isoleucine, phenylalanine, or tyrosine at a position selected from the group consisting of Q13, L19, R81, L85, S87, V91, I92, V93 and a combination thereof.

14. The modified IL-2 polypeptide of claim 13, which comprises a substitution with cysteine at a position selected from the group consisting of Q13, L19, R81, L85, S87, V91, I92, V93 and a combination thereof.

15. The modified IL-2 polypeptide of any of claims 6-14, which further comprises:
a) a substitution with a natural amino acid at a position within IL-2Rα interaction region and a substitution with a natural amino acid at a position within IL-2Rβ interaction region;
b) a substitution with a natural amino acid at a position within IL-2Rα interaction region and a substitution with a natural amino acid at a position within IL-2Rγ interaction region; or
c) a substitution with a natural amino acid at a position within IL-2Rα interaction region, a substitution with a natural amino acid at a position within IL-2Rβ interaction region and a substitution with a natural amino acid at a position within IL-2Rγ interaction region.

16. The modified IL-2 polypeptide of any of claims 1-15, which has reduced binding to an IL-2Rα compared to a comparable IL-2 polypeptide comprising an amino acid sequence set forth in SEQ ID NO:1 or SEQ ID NO:2 without the substitution.

17. The modified IL-2 polypeptide of claim 16, wherein its binding affinity to an IL-2Rα is decreased from about 10% to about 100%, or is decreased from about 1 fold to about 100,000 fold or more.

18. The modified IL-2 polypeptide of claim 16, which has no detectable binding to an IL-2Rα.

19. The modified IL-2 polypeptide of any of claims 1-18, which has reduced receptor signaling potency to IL-2αβγ compared to a comparable IL-2 polypeptide comprising an amino acid sequence set forth in SEQ ID NO:1 or SEQ ID NO:2 without the substitution.

20. The modified IL-2 polypeptide of claim 19, wherein a ratio between its signaling potency to IL-2αβγ and the signaling potency to IL-2αβγ of the comparable IL-2 polypeptide comprising an amino acid sequence set forth in SEQ ID NO:1 or SEQ ID NO:2 without the substitution is from about ½ to about 1/100,000.

21. The modified IL-2 polypeptide of claim 19, which has no detectable receptor signaling potency to IL-2Rαβγ.

22. The modified IL-2 polypeptide of any of claims 1-21, which has reduced binding to an IL-2Rα compared to a comparable IL-2 polypeptide comprising an amino acid sequence set forth in SEQ ID NO:1 or SEQ ID NO:2 without the substitution and has reduced receptor signaling potency to IL-2αβγ compared to a comparable IL-2 polypeptide comprising an amino acid sequence set forth in SEQ ID NO:1 or SEQ ID NO:2 without the substitution.

23. The modified IL-2 polypeptide of claim 22, which has no detectable binding to an IL-2Rα and has no detectable receptor signaling potency to IL-2αβγ.

24. The modified IL-2 polypeptide of any of claims 1-23, which retains substantial or has higher binding level to an interleukin 2 receptor β (IL-2Rβ) or an interleukin 2 receptor γ (IL-2Rγ) compared to a comparable IL-2 polypeptide comprising an amino acid sequence set forth in SEQ ID NO:1 or SEQ ID NO:2 without the substitution, and/or retains substantial or has higher receptor signaling potency to IL-2Rβγ compared to a comparable IL-2 polypeptide comprising an amino acid sequence set forth in SEQ ID NO:1 or SEQ ID NO:2 without the substitution.

25. The modified IL-2 polypeptide of claim 24, which retains substantial or has higher binding level to an IL-2Rβ or an IL-2Rγ compared to a comparable IL-2 polypeptide comprising an amino acid sequence set forth in SEQ ID NO:1 or SEQ ID NO:2 without the substitution.

26. The modified IL-2 polypeptide of claim 24, which retains substantial or has higher receptor signaling potency to IL-2Rβγ compared to a comparable IL-2 polypeptide comprising an amino acid sequence set forth in SEQ ID NO:1 or SEQ ID NO:2 without the substitution.

27. The modified IL-2 polypeptide of claim 24, which retains substantial or has higher binding level to an IL-2Rβ or an IL-2Rγ compared to a comparable IL-2 polypeptide comprising an amino acid sequence set forth in SEQ ID NO:1 or SEQ ID NO:2 without the substitution, and retains substantial or has higher receptor signaling potency to IL-2Rβγ compared to a comparable IL-2 polypeptide comprising an amino acid sequence set forth in SEQ ID NO:1 or SEQ ID NO:2 without the substitution.

28. The modified IL-2 polypeptide of claim 1-27, which has increased ratio of signaling potency to IL-2Rβγ over signaling potency to IL-2Rαβγ (i.e., increased ratio of signaling potency to IL-2Rβγ/signaling potency to IL-2Rαβγ) compared to a comparable IL-2 polypeptide comprising an amino acid sequence set forth in SEQ ID NO:1 or SEQ ID NO:2 without the substitution.

29. The modified IL-2 polypeptide of any of claims 1-28, which has a N terminal deletion, e.g., a N terminal deletion of amino acid residues 1-30, and/or a C terminal deletion, e.g., a C terminal deletion of amino acid residues 114-134.

30. The modified IL-2 polypeptide of any of claims 1-29, which has a N terminal deletion and a C terminal deletion.

31. The modified IL-2 polypeptide of any of claims 1-30, which is a part of a fusion polypeptide, e.g., a recombinant fusion protein, that comprises the modified IL-2 polypeptide and an additional amino acid sequence.

32. The modified IL-2 polypeptide of claim 31, wherein the N terminus or the C terminus of the modified IL-2 polypeptide is fused to the additional amino acid sequence.

33. The modified IL-2 polypeptide of claim 32, wherein the additional amino acid sequence comprises an antibody sequence or a portion or a fragment thereof, e.g., a Fc portion of an antibody.

34. The modified IL-2 polypeptide of any of claims 1-33, which is in an isolated form.

35. A polynucleotide, e.g., DNA, RNA or viral vector, that encodes a modified IL-2 polypeptide of any of claims 1-34 and is configured to express said modified IL-2 polypeptide in vitro and/or in vivo.

36. A modified IL-2 polypeptide conjugate, which comprises a modified IL-2 polypeptide of any of claim 1-34 or 1-35 that is conjugated to a water-soluble polymer, a lipid, a polypeptide, e.g., a protein, or a peptide.

37. The modified IL-2 polypeptide conjugate of claim 36, wherein the modified IL-2 polypeptide is conjugated to a water-soluble polymer, a lipid, a protein, or a peptide covalently.

38. The modified IL-2 polypeptide conjugate of claim 36, wherein the modified IL-2 polypeptide is conjugated to a water-soluble polymer, a lipid, a protein, or a peptide non-covalently.

39. The modified IL-2 polypeptide conjugate of any of claims 36-38, wherein the modified IL-2 polypeptide is conjugated to a water-soluble polymer, a lipid, a protein, or a peptide via a substituted natural amino acid or unnatural amino acid at a position selected from the group consisting of Q13, L19, N29, N30, Y31, K32, N33, P34, K35, T37, R38, T41, F42, K43, Y45, K48, K49, E62, K64, P65, N71, Q74, K76, R81, L85, S87, V91, I92, V93 and a combination thereof.

40. The modified IL-2 polypeptide conjugate of claim 39, wherein the modified IL-2 polypeptide is conjugated to a water-soluble polymer, a lipid, a protein, or a peptide via a substituted natural amino acid at a position selected from the group consisting of Q13, L19, N29, N30, Y31, K32, N33, P34, K35, T37, R38, T41, F42, K43, Y45, K48, K49, E62, K64, P65, N71, Q74, K76, R81, L85, S87, V91, I92, V93 and a combination thereof.

41. The modified IL-2 polypeptide conjugate of claim 39, wherein the modified IL-2 polypeptide is conjugated to a water-soluble polymer, a lipid, a protein, or a peptide via a substituted lysine, cysteine, histidine, arginine, aspartic acid, glutamic acid, serine, threonine, alanine, tryptophan, isoleucine, phenylalanine, or tyrosine at a position selected from the group consisting of Q13, L19, N29, N30, Y31, K32, N33, P34, K35, T37, R38, T41, F42, K43, Y45, K48, K49, E62, K64, P65, N71, Q74, K76, R81, L85, S87, V91, I92, V93 and a combination thereof.

42. The modified IL-2 polypeptide conjugate of claim 39, wherein the modified IL-2 polypeptide is conjugated to a water-soluble polymer, a lipid, a protein, or a peptide via a substituted cysteine at a position selected from the group consisting of Q13, L19, N29, N30, Y31, K32, N33, P34, K35, T37, R38, T41, F42, K43, Y45, K48, K49, E62, K64, P65, N71, Q74, K76, R81, L85, S87, V91, I92, V93 and a combination thereof.

43. The modified IL-2 polypeptide conjugate of claim 39, wherein the modified IL-2 polypeptide is conjugated to a water-soluble polymer, a lipid, a protein, or a peptide via a substituted natural amino acid or unnatural amino acid at a position selected from the group consisting of N29, N30, Y31, K32, N33, P34, K35, R38, T41, F42, K43, Y45, K48, K49, E62, K64, P65, N71, Q74, K76 and a combination thereof.

44. The modified IL-2 polypeptide conjugate of claim 43, wherein the modified IL-2 polypeptide is conjugated to a water-soluble polymer, a lipid, a protein, or a peptide via a substituted natural amino acid at a position selected from the group consisting of N29, N30, Y31, K32, N33, P34, K35, R38, T41, F42, K43, Y45, K48, K49, E62, K64, P65, N71, Q74, K76 and a combination thereof.

45. The modified IL-2 polypeptide conjugate of claim 44, wherein the modified IL-2 polypeptide is conjugated to a water-soluble polymer, a lipid, a protein, or a peptide via a substituted lysine, cysteine, histidine, arginine, aspartic acid, glutamic acid, serine, threonine, alanine, tryptophan, isoleucine, phenylalanine, or tyrosine at a position selected from the group consisting of N29, N30, Y31, K32, N33, P34, K35, R38, T41, F42, K43, Y45, K48, K49, E62, K64, P65, N71, Q74, K76 and a combination thereof.

46. The modified IL-2 polypeptide conjugate of claim 45, wherein the modified IL-2 polypeptide is conjugated to a water-soluble polymer, a lipid, a protein, or a peptide via a substituted cysteine at a position selected from the group consisting of N29, N30, Y31, K32, N33, P34, K35, R38, T41, F42, K43, Y45, K48, K49, E62, K64, P65, N71, Q74, K76 and a combination thereof.

47. The modified IL-2 polypeptide conjugate of any of claims 36-46, wherein the modified IL-2 polypeptide is conjugated to a water-soluble polymer, a lipid, a protein, or a peptide via a single amino acid residue of the modified IL-2 polypeptide.

48. The modified IL-2 polypeptide conjugate of claim 47, wherein the modified IL-2 polypeptide is conjugated to a water-soluble polymer, a lipid, a protein, or a peptide via:
i) the alpha amino group of the N-terminal amino acid residue of the modified IL-2 polypeptide;
ii) the epsilon amino group of a lysine amino acid residue of the modified IL-2 polypeptide; or
iii) an N-glycosylation site or O-glycosylation site of the modified IL-2 polypeptide.

49. The modified IL-2 polypeptide conjugate of any of claims 36-48, wherein the modified IL-2 polypeptide is covalently conjugated to a water-soluble polymer, a lipid, a protein, or a peptide through a linker.

50. The modified IL-2 polypeptide conjugate of any of claims 36-46, wherein the modified IL-2 polypeptide is conjugated to a water-soluble polymer, a lipid, a protein, or a peptide via a single amino acid residue in a fusion polypeptide that comprises the modified IL-2 polypeptide and an additional amino acid sequence.

51. The modified IL-2 polypeptide conjugate of claim 50, wherein the single amino acid residue is located within the modified IL-2 polypeptide.

52. The modified IL-2 polypeptide conjugate of claim 50, wherein the single amino acid residue is located within the additional amino acid sequence.

53. The modified IL-2 polypeptide conjugate of any of claims 50-52, wherein the additional amino acid sequence comprises an antibody sequence or a portion or a fragment thereof.

54. The modified IL-2 polypeptide conjugate of claim 53, wherein the additional amino acid sequence comprises a Fc portion of an antibody.

55. The modified IL-2 polypeptide conjugate of any of claims 50-54, wherein the modified IL-2 polypeptide is conjugated to a water-soluble polymer, a lipid, a protein, or a peptide via:
i) the alpha amino group of the N-terminal amino acid residue of the fusion polypeptide;
ii) the epsilon amino group of a lysine amino acid residue of the fusion polypeptide; or
iii) an N-glycosylation site or O-glycosylation site of the fusion polypeptide.

56. The modified IL-2 polypeptide conjugate of claim 55, wherein the fusion polypeptide is covalently conjugated to a water-soluble polymer, a lipid, a protein, or a peptide through a linker.

57. The modified IL-2 polypeptide conjugate of any of claims 36-56, wherein the modified IL-2 polypeptide is conjugated to a water-soluble polymer.

58. The modified IL-2 polypeptide conjugate of claim 47, wherein the water-soluble polymer comprises polyethylene glycol (PEG), poly(propylene glycol) (PPG), copolymers of ethylene glycol and propylene glycol, poly(oxyethylated polyol), poly(olefinic alcohol), poly(vinylpyrrolidone), poly(hydroxyalkylmethacrylamide), poly(hydroxyalkylmethacrylate), poly(saccharides), poly(a-hydroxy acid), poly(vinyl alcohol), polyphosphazene, polyoxazolines (POZ), poly(N-acryloylmorpholine), or a combination thereof.

59. The modified IL-2 polypeptide conjugate of claim 58, wherein the water-soluble polymer comprises a PEG molecule.

60. The modified IL-2 polypeptide conjugate of claim 59, wherein the PEG molecule is a linear PEG.

61. The modified IL-2 polypeptide conjugate of claim 59, wherein the PEG molecule is a branched PEG.

62. The modified IL-2 polypeptide conjugate of claim 61, wherein the branched PEG has about three to about ten PEG chains emanating from a central core group.

63. The modified IL-2 polypeptide conjugate of claim 61, wherein the branched PEG is a star PEG comprising from about 10 to about 100 PEG chains emanating from a central core group.

64. The modified IL-2 polypeptide conjugate of claim 61, wherein the branched PEG is a comb PEGs comprising multiple PEG chains grafted onto a polymer backbone.

65. The modified IL-2 polypeptide conjugate of any of claims 59-64, wherein the PEG molecule has a range of molecular weight from about 300 g/mol to about 10,000,000 g/mol.

66. The modified IL-2 polypeptide conjugate of any of claims 59-64, wherein the PEG molecule has an average molecular weight from about 5,000 Daltons to about 1,000,000 Daltons.

67. The modified IL-2 polypeptide conjugate of claim 66, wherein the PEG molecule has an average molecular weight of from about 20,000 Daltons to about 30,000 Daltons.

68. The modified IL-2 polypeptide conjugate of any of claims 59-67, wherein the PEG molecule is a monodisperse, uniform, or discrete PEG molecule.

69. The modified IL-2 polypeptide conjugate of claim 57, wherein the water-soluble polymer comprises a polysaccharide.

70. The modified IL-2 polypeptide conjugate of any of claims 36-56, wherein the modified IL-2 polypeptide is conjugated to a lipid.

71. The modified IL-2 polypeptide conjugate of claim 70, wherein the lipid comprises a fatty acid.

72. The modified IL-2 polypeptide conjugate of any of claims 36-56, wherein the modified IL-2 polypeptide is conjugated to a protein.

73. The modified IL-2 polypeptide conjugate of claim 72, wherein the protein comprises an antibody or a binding fragment thereof.

74. The modified IL-2 polypeptide conjugate of claim 73, wherein the antibody or a binding fragment thereof comprises an Fc portion of an antibody.

75. The modified IL-2 polypeptide conjugate of any of claims 36-74, wherein a water-soluble polymer, a lipid, a protein, or a peptide is indirectly bound to the substituted natural amino acid or unnatural amino acid of the modified IL-2 polypeptide through a linker.

76. The modified IL-2 polypeptide conjugate of any of claims 36-74, wherein a water-soluble polymer, a lipid, a protein, or a peptide is directly bound to the substituted natural amino acid or unnatural amino acid of the modified IL-2 polypeptide.

77. The modified IL-2 polypeptide conjugate of any of claims 36-76, which has a half-life in vivo from about 5 minutes to about 10 days.

78. A pharmaceutical composition comprising an effective amount of a modified IL-2 polypeptide of any of claims 1-34, a polynucleotide of claim 35 or a modified IL-2 polypeptide conjugate of any of claims 36-77, and a pharmaceutically acceptable carrier or excipient.

79. The pharmaceutical composition of claim 78, which further comprises another active ingredient.

80. The pharmaceutical composition of claim 78 or 79, which is configured to treat or prevent a proliferation disorder in a subject.

81. The pharmaceutical composition of claim 79, wherein the another active ingredient is an anti-neoplasm substance.

82. A method for treating or preventing a disease or a disorder, e.g., a proliferation disease or disorder, an autoimmune or inflammatory disease or disorder, or an infectious disease or disorder, in a subject in need comprising administering to said subject an effective amount of a modified IL-2 polypeptide of any of claims 1-34, a polynucleotide of claim 35, a modified IL-2 polypeptide conjugate of any of claims 36-77, or a pharmaceutical composition of any of claims 78-81.

83. The method of claim 82, wherein the subject s a human.

84. The method of claim 82, wherein the subject is a non-human mammal.

85. The method of any of claims 82-84, which is used to treat a proliferation disorder in a subject.

86. The method of any of claims 82-84, which is used to prevent a proliferation disorder in a subject.

87. The method of any of claims 82-86, wherein the proliferation disorder is a tumor.

88. The method of any of claims 82-86, wherein the proliferation disorder is a cancer.

89. The method of claim 87 or 88, wherein the tumor or cancer is a solid tumor or cancer.

90. The method of claim 89, wherein the solid tumor or cancer is selected from the group consisting of: Chondrosarcoma, Ewing's sarcoma, Malignant fibrous histiocytoma of bone/osteosarcoma, Osteosarcoma, Rhabdomyosarcoma, Heart cancer, Astrocytoma, Brainstem glioma, Pilocytic astrocytoma, Ependymoma, Primitive neuroectodermal tumor, Cerebellar astrocytoma, Cerebral astrocytoma, Glioma, Medulloblastoma, Neuroblastoma, Oligodendroglioma, Pineal astrocytoma, Pituitary adenoma, Visual pathway and hypothalamic glioma, Breast cancer, Invasive lobular carcinoma, Tubular carcinoma, Invasive cribriform carcinoma, Medullary carcinoma, Male breast cancer, Phyllodes tumor, Inflammatory Breast Cancer, Adrenocortical carcinoma, Islet cell carcinoma (endocrine pancreas), Multiple endocrine neoplasia syndrome, Parathyroid cancer, Pheochromocytoma, Thyroid cancer, Merkel cell carcinoma, Uveal melanoma, Retinoblastoma, Anal cancer, Appendix cancer, cholangiocarcinoma, Carcinoid tumor, gastrointestinal, Colon cancer, Extrahepatic bile duct cancer, Gallbladder cancer, Gastric (stomach) cancer, Gastrointestinal carcinoid tumor, Gastrointestinal stromal tumor (GIST), Hepatocellular cancer, Pancreatic cancer islet cell, Rectal cancer, Bladder cancer, Cervical cancer, Endometrial cancer, Extragonadal germ cell tumor, Ovarian cancer, Ovarian epithelial cancer (surface epithelial-stromal tumor), Ovarian germ cell tumor, Penile cancer, Renal cell carcinoma, Renal pelvis and ureter, transitional cell cancer, Prostate cancer, Testicular cancer, Gestational trophoblastic tumor, Ureter and renal pelvis, transitional cell cancer, Urethral cancer, Uterine sarcoma, Vaginal cancer, Vulvar cancer, Wilms tumor, Esophageal cancer, Head and neck cancer, Nasopharyngeal carcinoma, Oral cancer, Oropharyngeal cancer, Paranasal sinus and nasal cavity cancer, Pharyngeal cancer, Salivary gland cancer, Hypopharyngeal cancer, Basal-cell carcinoma, Melanoma, Skin cancer (non-melanoma), Bronchial adenomas/carcinoids, Small cell lung cancer, Mesothelioma, Non-small cell lung cancer, Pleuropulmonary blastoma, Laryngeal cancer, Thymoma and thymic carcinoma, AIDS-related cancers, Kaposi sarcoma, Epithelioid hemangioendothelioma (EHE), Desmoplastic small round cell tumor and Liposarcoma.

91. The method of claim 87 or 88, wherein the tumor or cancer is a hematological malignancy.

92. The method of claim 91, wherein the hematological malignancy is selected from the group consisting of: myeloid neoplasms, Leukemias, Lymphomas, Hodgkin lymphoma, Non-Hodgkin lymphoma, Anaplastic large cell lymphoma, Angioimmunoblastic T-cell lymphoma, Hepatosplenic T-cell lymphoma, B-cell lymphoma reticuloendotheliosis, Reticulosis, Microglioma, Diffuse large B-cell lymphoma, Follicular lymphoma, Mucosa-associated lymphatic tissue lymphoma, B-cell chronic lymphocytic leukemia, Mantle cell lymphoma, Burkitt lymphoma, Mediastinal large B cell lymphoma, Waldenström's macroglobulinemia, Nodal marginal zone B cell lymphoma, Splenic marginal zone lymphoma, Intravascular large B-cell lymphoma, Primary effusion lymphoma, Lymphomatoid granulomatosis, Nodular lymphocyte predominant Hodgkin's lymphoma, plasma cell leukemia, Acute erythraemia and erythroleukaemia, Acute erythremic myelosis, Acute erythroid leukemia, Heilmeyer-Schoner disease, Acute megakaryoblastic leukemia, Mast cell leukemia, Panmyelosis, Acute panmyelosis with myelofibrosis, Lymphosarcoma cell leukemia, Acute leukaemia of unspecified cell type, Blastic phase chronic myelogenous leukemia, Stem cell leukemia, Chronic leukaemia of unspecified cell type, Subacute leukaemia of unspecified cell type, Accelerated phase chronic myelogenous leukemia, Acute myeloid leukemia, Polycythemia vera, Acute promyelocytic leukemia, Acute basophilic leukemia, Acute eosinophilic leukemia, Acute lymphoblastic leukemia, Acute monocytic leukemia, Acute myeloblastic leukemia with maturation, Acute myeloid dendritic cell leukemia, Adult T-cell leukemia/lymphoma, Aggressive NK-cell leukemia, B-cell prolymphocytic leukemia, B-cell chronic lymphocytic leukemia, B-cell leukemia, Chronic myelogenous leukemia, Chronic myelomonocytic leukemia, Chronic neutrophilic leukemia, Chronic lymphocytic leukemia, Hairy cell leukemia, Chronic idiopathic myelofibrosis, Multiple myeloma, Kahler's disease, Myelomatosis, Solitary myeloma, Plasma cell leukemia, Plasmacytoma, extramedullary, Malignant plasma cell tumour NOS, Plasmacytoma NOS, Monoclonal gammopathy, Multiple Myeloma, Angiocentric immunoproliferative lesion, Lymphoid granulomatosis, Angioimmunoblastic lymphadenopathy, T-gamma lymphoproliferative disease, Waldenström's macroglobulinaemia, Alpha heavy chain disease, Gamma heavy chain disease, Franklin's disease, Immunoproliferative small intestinal disease, Mediterranean disease, Malignant immunoproliferative disease, unspecified, and Immunoproliferative disease NOS.

93. The method of claim 82, wherein the disease or disorder is an immune deficiency disease or disorder.

94. The method of claim 93, wherein the immune deficiency disease or disorder is selected from the group consisting of: Agammaglobulinemia: X-Linked and Autosomal Recessive, Ataxia Telangiectasia, Chronic Granulomatous Disease and Other Phagocytic Cell Disorders, Common Variable Immune Deficiency, Complement Deficiencies, DiGeorge Syndrome, Hemophagocytic Lymphohistiocytosis (HLH), Hyper IgE Syndrome, Hyper IgM Syndromes, IgG Subclass Deficiency, Innate Immune Defects, NEMO Deficiency Syndrome, Selective IgA Deficiency, Selective IgM Deficiency, Severe Combined Immune, Deficiency and Combined Immune Deficiency, Specific Antibody Deficiency, Transient Hypogammaglobulinemia of Infancy, WHIM Syndrome (Warts, Hypogammaglobulinemia, Infections, and Myelokathexis), Wiskott-Aldrich Syndrome, Other Antibody Deficiency Disorders, Other Primary Cellular Immunodeficiencies, Severe combined immune deficiency (SCID), Common variable immune deficiency (CVID), Human immunodeficiency virus/acquired immune deficiency syndrome (HIV/AIDS), Drug-induced immune deficiency, Graft versus host syndrome, Primary Immune Deficiency Diseases (PIDDs), and Lymphopenia.

95. The method of any of claims 82-84, which is used to treat or prevent an autoimmune or inflammatory disease or disorder in a subject.

96. The method of claim 95, wherein the inflammatory or autoimmune diseases or disorder is selected from the group consisting of: inflammation, autoimmune disease, paraneoplastic autoimmune diseases, cartilage inflammation, fibrotic disease and/or bone degradation, arthritis, rheumatoid arthritis, juvenile arthritis, juvenile rheumatoid arthritis, pauciarticular juvenile rheumatoid arthritis, polyarticular juvenile rheumatoid arthritis, systemic onset juvenile rheumatoid arthritis, juvenile ankylosing spondylitis, juvenile enteropathic arthritis, juvenile reactive arthritis, juvenile Reter's Syndrome, SEA Syndrome (Seronegativity, Enthesopathy, Arthropathy Syndrome), juvenile dermatomyositis, juvenile psoriatic arthritis, juvenile scleroderma, juvenile systemic lupus erythematosus, juvenile vasculitis, pauciarticular rheumatoid arthritis, polyarticular rheumatoid arthritis, systemic onset rheumatoid arthritis, ankylosing spondylitis, enteropathic arthritis, reactive arthritis, Reter's Syndrome, SEA Syndrome (Seronegativity, Enthesopathy, Arthropathy Syndrome), dermatomyositis, psoriatic arthritis, scleroderma, systemic lupus erythematosus, vasculitis, myolitis, polymyolitis, dermatomyolitis, osteoarthritis, polyarteritis nodossa, Wegener's granulomatosis, arteritis, ploymyalgia rheumatica, sarcoidosis, scleroderma, sclerosis, primary biliary sclerosis, sclerosing cholangitis, Sjogren's syndrome, psoriasis, plaque psoriasis, guttate psoriasis, inverse psoriasis, pustular psoriasis, erythrodermic psoriasis, dermatitis, atopic dermatitis, atherosclerosis, lupus, Still's disease, Systemic Lupus Erythematosus (SLE), myasthenia gravis, inflammatory bowel disease (IBD), Crohn's disease, ulcerative colitis, celiac disease, multiple schlerosis (MS), asthma, COPD, Guillain-Barre disease, Type I diabetes mellitus, thyroiditis (e.g., Graves' disease), Addison's disease, Raynaud's phenomenon, autoimmune hepatitis, GVHD, and transplantation rejection.

97. The method of any of claims 82-84, which is used to treat or prevent an infectious disease or disorder in a subject.

98. The method of claim 97, wherein the infectious diseases is selected from the group consisting of: Acinetobacter infections, Actinomycosis, African sleeping sickness (African trypanosomiasis), AIDS (acquired immunodeficiency syndrome), Amoebiasis, Anaplasmosis, Angiostrongyliasis Anisakiasis, Anthrax, Arcanobacterium haemolyticum infection, Argentine hemorrhagic fever, Ascariasis, Aspergillosis Astrovirus infection, Babesiosis, Bacillus cereus infection, Bacterial meningitis, Bacterial pneumonia, Bacterial vaginosis, Bacteroides infection, Balantidiasis, Bartonellosis, Baylisascaris infection, BK virus infection, Black piedra, Blastocystosis, Blastomycosis, Bolivian hemorrhagic fever, Botulism (and Infant botulism), Brazilian hemorrhagic fever, Brucellosis, Bubonic plague, Burkholderia infection, Buruli ulcer, Calicivirus infection (Norovirus and Sapovirus), Campylobacteriosis, Candidiasis (Moniliasis; Thrush), Capillariasis, Carrion's disease, Cat-scratch disease, Cellulitis, Chagas disease (American trypanosomiasis), Chancroid, Chickenpox, Chikungunya, Chlamydia, Chlamydophila pneumoniae infection (Taiwan acute respiratory agent or TWAR), Cholera, Chromoblastomycosis, Chytridiomycosis, Clonorchiasis, Clostridium difficile colitis, Coccidioidomycosis, Colorado tick fever (CTF), Common cold (Acute viral rhinopharyngitis; Acute coryza), Coronavirus disease 2019 (COVID-19), Creutzfeldt-Jakob disease (CJD), Crimean-Congo hemorrhagic fever (CCHF), Cryptococcosis, Cryptosporidiosis, Cutaneous larva migrans (CLM), Cyclosporiasis, Cysticercosis, Cytomegalovirus infection, Dengue fever, Desmodesmus infection, Dientamoebiasis, Diphtheria, Diphyllobothriasis, Dracunculiasis, Ebola hemorrhagic fever, Echinococcosis, Ehrlichiosis, Enterobiasis (Pinworm infection), Enterococcus infection, Enterovirus infection, Epidemic typhus, Erythema infectiosum (Fifth disease), Exanthem subitum (Sixth disease), Fasciolasis, Fasciolopsiasis, Fatal familial insomnia (FFI), Filariasis, Food poisoning by Clostridium perfringens, Free-living amebic infection, Fusobacterium infection, Gas gangrene (Clostridial myonecrosis), Geotrichosis, Gerstmann-Sträussler-Scheinker syndrome (GSS), Giardiasis, Glanders, Gnathostomiasis, Gonorrhea, Granuloma inguinale (Donovanosis), Group A streptococcal infection, Group B streptococcal infection, Haemophilus influenzae infection, Hand, foot and mouth disease (HFMD), Hantavirus Pulmonary Syndrome (HPS), Heartland virus disease, Helicobacter pylori infection, Hemolytic-uremic syndrome (HUS), Hemorrhagic fever with renal syndrome (HFRS), Hendra virus infection, Hepatitis A, Hepatitis B, Hepatitis C, Hepatitis D, Hepatitis E, Herpes simplex, Histoplasmosis, Hookworm infection, Human bocavirus infection, Human ewingii ehrlichiosis, Human granulocytic anaplasmosis (HGA), Human metapneumovirus infection, Human monocytic ehrlichiosis, Human papillomavirus (HPV) infection, Human parainfluenza virus infection, Hymenolepiasis, Epstein-Barr virus infectious mononucleosis (Mono), Influenza (flu), Isosporiasis, Kawasaki disease, Keratitis, Kingella kingae infection, Kuru, Lassa fever, Legionellosis (Legionnaires' disease), Pontiac fever, Leishmaniasis, Leprosy, Leptospirosis, Listeriosis, Lyme disease (Lyme borreliosis), Lymphatic filariasis (Elephantiasis), Lymphocytic choriomeningitis, Malaria, Marburg hemorrhagic fever (MHF), Measles, Middle East respiratory syndrome (MERS), Melioidosis (Whitmore's disease), Meningitis, Meningococcal disease, Metagonimiasis, Microsporidiosis, Molluscum contagiosum (MC), Monkeypox, Mumps, Murine typhus (Endemic typhus), Mycoplasma pneumonia, Mycoplasma genitalium infection, Mycetoma, Myiasis, Neonatal conjunctivitis (Ophthalmia neonatorum), Nipah virus infection, Norovirus (children and babies), (New) Variant Creutzfeldt-Jakob disease (vCJD, nvCJD), Nocardiosis, Onchocerciasis (River blindness), Opisthorchiasis, Paracoccidioidomycosis (South American blastomycosis), Paragonimiasis, Pasteurellosis, Pediculosis capitis (Head lice), Pediculosis corporis (Body lice), Pediculosis pubis (pubic lice, crab lice), Pelvic inflammatory disease (PID), Pertussis (whooping cough), Plague, Pneumococcal infection, Pneumocystis pneumonia (PCP), Pneumonia, Poliomyelitis, Prevotella infection, Primary amoebic meningoencephalitis (PAM), Progressive multifocal leukoencephalopathy, Psittacosis, Q fever, Rabies, Relapsing fever, Respiratory syncytial virus infection, Rhinosporidiosis, Rhinovirus infection, Rickettsial infection, Rickettsialpox, Rift Valley fever (RVF), Rocky Mountain spotted fever (RMSF), Rotavirus infection, Rubella, Salmonellosis, SARS (severe acute respiratory syndrome), Scabies, Scarlet fever, Schistosomiasis, Sepsis, Shigellosis (bacillary dysentery), Shingles (Herpes zoster), Smallpox (variola), Sporotrichosis, Staphylococcal food poisoning, Staphylococcal infection, Strongyloidiasis, Subacute sclerosing panencephalitis, Bejel, Syphilis, and Yaws, Taeniasis, Tetanus (lockjaw), Tinea barbae (barber's itch), Tinea capitis (ringworm of the scalp), Tinea corporis (ringworm of the body), Tinea cruris (Jock itch), Tinea manum (ringworm of the hand), Tinea nigra, Tinea pedis (athlete's foot), Tinea unguium (onychomycosis), Tinea versicolor (Pityriasis versicolor), Toxocariasis (ocular larva migrans (OLM)), Toxocariasis (visceral larva migrans (VLM)), Toxoplasmosis, Trachoma, Trichinosis, Trichomoniasis, Trichuriasis (whipworm infection), Tuberculosis, Tularemia, Typhoid fever, Typhus fever, Ureaplasma urealyticum infection, Valley fever, Venezuelan equine encephalitis, Venezuelan hemorrhagic fever, Vibrio vulnificus infection, Vibrio parahaemolyticus enteritis, Viral pneumonia, West Nile fever, White piedra (tinea blanca), Yersinia pseudotuberculosis infection, Yersiniosis, Yellow fever, Zeaspora, Zika fever, and Zygomycosis.

99. The method of any of claims 82-99, which further comprises administering an effective amount of a second therapeutic agent for treating or preventing a proliferation disorder in a subject.

100. Use of an effective amount of a modified IL-2 polypeptide of any of claims 1-34, a polynucleotide of claim 35 or a modified IL-2 polypeptide conjugate of any of claims 36-77 for the manufacture of a medicament for treating or preventing a disease or a disorder, e.g., a proliferation disease or disorder, an autoimmune or inflammatory disease or disorder, or an infectious disease or disorder, in a subject.

101. A method of expanding a CD4⁺ helper cell, CD8⁺ effector naive and memory cell, Natural Killer (NK) cell, or Natural killer T (NKT) cell population, which comprises contacting a cell population with an effective amount of a modified IL-2 polypeptide of any of claims 1-34, a polynucleotide of claim 35, a modified IL-2 polypeptide conjugate of any of claims 36-77, or a pharmaceutical composition of any of claims 78-81 for a time sufficient to induce formation of a complex with an IL-2Rβγ, thereby stimulating the expansion of the T cell, NK cell, and/or NKT cell population.

102. A method of expanding a CD4⁺ helper cell, CD8⁺ effector naive and memory cell, Treg cells, Natural Killer (NK) cell, or Natural killer T (NKT) cell population, which comprises contacting a cell population with an effective amount of a modified IL-2 polypeptide of any of claims 1-34, a polynucleotide of claim 35, a modified IL-2 polypeptide conjugate of any of claims 36-77, or a pharmaceutical composition of any of claims 78-81 for a time sufficient to induce formation of a complex with an IL-2Rβγ, thereby stimulating the expansion of the T cell, Treg cell, NK cell, and/or NKT cell population with reduced cell death by 10% to 100%.

103. The method of claim 101 or 102, wherein the modified IL-2 polypeptide of any of claims 1-34, a polynucleotide of claim 35, a modified IL-2 polypeptide conjugate of any of claims 36-77, or a pharmaceutical composition of any of claims 78-81 expands CD4⁺ T regulatory (Treg) cells by less than 20%, 15%, 10%, 5%, 1% or less in the CD3⁺ cell population compared to an expansion of CD4⁺ Treg cells in the CD3⁺ cell population contacted with a comparable IL-2 polypeptide comprising an amino acid sequence set forth in SEQ ID NO:1 or SEQ ID NO:2 without the substitution.

104. The method of claim 101 or 102, wherein the modified IL-2 polypeptide of any of claims 1-34, the polynucleotide of claim 35, the modified IL-2 polypeptide conjugate of any of claims 36-77, or the pharmaceutical composition of any of claims 78-81 does not expand CD4⁺ Treg cells in the cell population.

105. The method of claim 101 or 102, wherein the ratio of the Teff cells to Treg cells in the cell population after incubation with the modified IL-2 polypeptide of any of claims 1-34, the polynucleotide of claim 35, the modified IL-2 polypeptide conjugate of any of claims 36-77, or the pharmaceutical composition of any of claims 78-81 is about or at least 2:1, 3:1, 4:1, 5:1, 6:1, 7:1, 8:1, 9:1, 10:1, 20:1, 50:1, 100:1 or more.

106. The method of any of claims 101-105, which is conducted in vivo.

107. The method of any of claims 101-105, which is conducted in vitro.

108. The method of any of claims 101-105, which is conducted ex vivo.

109. Use of an effective amount of a modified IL-2 polypeptide of any of claims 1-34, a polynucleotide of claim 35, or a modified IL-2 polypeptide conjugate of any of claims 36-77 for the manufacture of a medicament for expanding a CD4⁺ helper cell, CD8⁺ effector naive and memory cell, Treg cell, Natural Killer (NK) cell, or Natural killer T (NKT) cell population in a cell population.

110. The use of claim 109, which is configured for expanding a CD4⁺ helper cell, CD8⁺ effector naive and memory cell, Treg cell, Natural Killer (NK) cell, or Natural killer T (NKT) cell population in a subject.
